(19) United States
(12) Patent Application Publication
(10) Pub. No.: US 2025/0368776 A1
(43) Pub. Date: Dec. 4, 2025

(54) MULTIFUNCTIONAL POLYMER AND COMPOSITION FOR TISSUE REGENERATION AND METHODS THEREOF

(71) Applicant: THE UNIVERSITY OF HONG KONG, Hong Kong (CN)

(72) Inventors: Waruna Lakmal DISSANAYAKA, Hong Kong (CN); Shuting Gao, Hong Kong (CN); Huihua Li, Hong Kong (CN); Jian He, Hong Kong (CN)

(73) Assignee: THE UNIVERSITY OF HONG KONG, Hong Kong (CN)

(21) Appl. No.: 18/678,375

(22) Filed: May 30, 2024

(51) Int. Cl. C08G 63/12; A61K 6/30; A61K 31/765; A61P 1/02; C08G 63/91

(52) U.S. Cl. CPC C08G 63/12; A61K 6/30; A61K 31/765; A61P 1/02; C08G 63/914

(57) ABSTRACT

The present disclosure provides a biocompatible polymer and composition having antibacterial and osteogenic properties to promote periodontal tissue regeneration. The polymer is a polyxylitol succinate (PXS) polymer prepared by esterifying xylitol with succinyl chloride. Adhesive properties were subsequently incorporated into the polymer by combining biocompatible catechol via caffeic acid (CFA) in the presence of citric acid. The final injectable caffeic acid/PXS/citric acid composite polymer (iCPC) hydrogel was fabricated by cross-linking the pre-polymer solution with MgO. PXS and iCPC polymers have anti-bacterial effects and osteogenic properties. The disclosure further provides uses of the polymer such as tissue engineering, drug delivery, as a bioadhesive in wound healing, as bone substitutes or scaffolds, as cements in dental and periodontal applications and as anti-adhesives or protective barriers.

### 1. FIELD

The disclosure is generally directed to a bioadhesive injectable polymer with anti-inflammatory, antibacterial, and osteogenic properties to promote tissue regeneration.

### 2. BACKGROUND

Tissue adhesives have been introduced as a promising alternative to traditional wound suturing methods. Designing and developing tissue adhesives for biomedical applications have been a research focus for decades. Adhesive materials possess an array of unique chemical functionalities in elastomers and adhesives, which make them useful in a wide variety of biomaterial applications such as drug delivery^{[1]}, dental materials[²], biosensors^{[3]}, and tissue engineering^{[4]}. Both biologic adhesives (e.g., fibrinogen-based) [5] and synthetic adhesives (e.g., cyanoacrylate-based adhesives)^{[6]} are available for such applications. However, most synthetic adhesives are polymer-based, especially poly (ethylene glycol) (PEG) based bioadhesives, such as CoSeal and DuraSeal, their bioavailability and bioactivity are limited by immune and inflammatory responses. For example, about 20-70% of humans have an anti-PEG immune response that can reduce the effectiveness of certain PEGylated therapeutics^{[7]}. Additionally, most synthetic adhesives and their degradation products lead to a pro-inflammatory response during the decomposition process, such concerns are observed in the clinical application of PLGA, where serious inflammatory reactions were reported^{[8]}. Such a dysregulated immune and inflammatory response can cause a catastrophic cascade characterized by local or systemic tissue damage and excessive danger signal production, ultimately leading to tissue regeneration failure. To tackle this problem, numerous researchers have focused on enhancing polymer-based adhesives to attain anti-inflammatory effects, typically employing delivery methods including chemo drugs, biomacromolecules (nucleic acids and proteins), and therapeutic gas^{[9-10]}. However, their bioapplication is difficult to achieve due to poor water solubility and off-target toxicity, which are the major causes of failure in clinical trials^{[11-13]}. As a result, there are still challenges in achieving anti-inflammatory functionality in polymeric-based adhesives. Consequently, rather than concentrating solely on the loaded agents, these concerns could be addressed by using and modifying polymers with inherent anti-inflammatory properties, which reduce the need for additional agents and their potential side effects to optimize the performance and applicability of polymer in hydrogel applications. Although, compared with synthetic adhesives, biologic adhesives tend to have higher biocompatibility, and their biodegradation is less likely to cause tissue irritation^{[14]}, when facing complex treatments or application needs, these types of natural adhesives usually fail to endow these bioadhesives with specific biological functions. For example, fibrin glue has anti-inflammatory functions but lacks antibacterial and bone regeneration properties^{[15]}. Therefore, a multifunctional adhesive system with anti-inflammatory, osteogenic, and antibacterial capabilities is urgently needed to treat inflammatory diseases such as periodontitis, rheumatoid arthritis, or osteomyelitis.

Xylitol-based polymers have shown excellent biodegradability, biocompatibility, and low cytotoxicity both in vitro and in vivo. Research has also explored various polyesters, e.g., xylitol-sebacic acid^{[16]}, xylitol-glutamic acid^{[17]}, and xylitol-succinate acid^{[18]}. However, little has been done to develop xylitol-based bioadhesive hydrogels so far. In dentistry, xylitol is also well known to promote oral health by reducing 20% of dental plaque accumulation and inhibiting the P. gingivalis and Actinobacteria growth, which are the pathogenic bacteria in periodontitis^{[19,20]}. Periodontal disease (PD) is the 11^{th} most prevalent disease among adults, affecting 20-50% of the global adult population^{[21]}. Current clinical management of PD is primarily based on disinfection through flap debridement and/or curettage and tissue regenerative strategies with periodontal membranes and grafting materials^{[22]}. Strong adhesion to the root surface with surrounding soft gingival tissues in the wet state is a prerequisite for improvement in therapeutic efficacy that can be achievable through the polymer-based bio-adhesive hydrogel. Caffeic acid (3,4-dihydroxy-cinnamic acid), on the other hand, is a natural compound found in various plant sources, including coffee, fruits, and vegetables. Also, it is an example of a catecholic organic compound that could provide adhesive properties to a polymer. Beyond that, the outstanding performances of caffeic acid include anti-inflammatory in periodontal tissue^{[23]}, antibacterial activity^{[24]}, antioxidant properties^{[25]}, and prevention of a variety of cancers by metabolism interference or apoptosis induction^{[26,27]}. To enhance the weak adhesive properties of natural adhesives, citric acid has been used as a safe crosslinking agent. This is because it is cost-effective, non-toxic, and has hydrophilic properties. Additionally, as a natural organic compound with 3-OH groups, it can form a network in most hydrogel formulations^{[28]}. A significant aspect of citric acid-derived adhesives is that citric acid plays a valuable role in the formation of ester bond-crosslinks, improving tensile strength, balancing the hydrophilicity of the polymer network, and providing hydrogen bonding and additional binding sites for bioconjugation to create a strongly crosslinked hydrogel network^{[29]}.

In addition to the requirements of anti-bacterial properties to bioadhesives in periodontitis therapy, the effective osteoinductive nature remains equally critical, especially at the later stage of inflammation. In many reports, magnesium ions (Mg²⁺) have been proven to have excellent osteogenic properties^{[30,31]}. Mg²⁺ has also been dedicated to improving the mechanical properties of hydrogels or shortening the gelation time as a crosslinker^{[32,33]}. Furthermore, an eco-friendly synthetic route is essential for the preparation of biomaterials, minimizing the use of toxic reagents.

Therefore, a bioadhesive with excellent biological properties, including antibacterial, anti-inflammatory, and osteogenic properties, can be developed by combining citric acid, xylitol-based polymer, and caffeic acid.

### 3. SUMMARY

To overcome the limitations of existing polymer-based hydrogels and to efficiently bind the series of bio-advantages of xylitol, caffeic acid, and magnesium in achieving a multifunctional bioadhesive, more importantly, to meet the clinical demand for multifunctional materials. We have designed a novel injectable xylitol-based polymer hydrogel with tissue adhesive characteristics to assist regenerative treatments via two steps of chemical synthesis and one step of cross-linking. This new polymer may have a broadened range of applications in other fields. Compared with the traditional chitosan or other polymer-dependent hydrogels, our adhesive system makes it easier to meet the biological function requirements of dental therapy, what's more, the natural materials that we use are much more sustainable and environmentally friendly.

The disclosure provides the polymer and its characterization in biomedical applications. The disclosure further provides methods of using polymer compositions such as in tissue engineering, drug delivery, as a bioadhesive in wound healing, as bone substitutes or scaffolds, as cements in dental and periodontal applications. In one embodiment, provided herein is a method of treating periodontitis using the polymer and composition disclosed herein.

In one embodiment, polymers are provided that are biocompatible and biodegradable. In one embodiment, the polymers are suitable for use in medical applications.

In one embodiment, provided are polymers with tunable properties.

In one embodiment, provided is an injectable adhesive hydrogel with antibacterial and osteogenic properties to promote periodontal regeneration.

In one embodiment, provided herein is a caffeic acid modified Poly (xylitol Succinate)-citric acid polymer cross-linked by magnesium oxide. In one embodiment, the hydrogel is a multifunctional injectable system healing periodontitis.

In one embodiment, provided are prepolymers and polymers based on xylitol and succinyl chloride monomers, as well as composites containing such polymers.

In one embodiment, provided are prepolymers and polymers based on xylitol and succinyl chloride monomers combining with a biocompatible catechol.

In one embodiment the catechol is caffeic acid (CFA).

In one embodiment, the PXS and citric acid were first 160° C. for 1 h under nitrogen protection, then added CFA to form iCPC.

In one embodiment, the injectable CFA/PXS/Citric acid (“iCPC”) hydrogel was formed by cross-linking the pre-polymer solution with MgO.

In one embodiment, provided herein is an iCPC hydrogel. In one embodiment, the hydrogel possesses excellent tissue-adhesive, antibacterial, and osteogenic properties and stimulates periodontal regeneration in vivo.

In one embodiment, provided herein, is a polyxylitol succinate (PXS) polymer prepared by esterifying xylitol with succinyl chloride. In one embodiment, the PXS is mixed with citric acid, followed by the addition of caffeic acid (CFA) in a one-pot synthesis. In one embodiment, bioadhesive properties are obtained by adding catechol moieties containing CFA. In one embodiment, the final injectable caffeic acid/PXS/citric acid composite (iCPC) hydrogel is fabricated by cross-linking the polymer solution with MgO. In one embodiment, the PXS and iCPC polymers are characterized by proton nuclear magnetic resonance (1H NMR) spectra; adhesiveness, biocompatibility, and biodegradation are optimized; intrinsic anti-bacterial effects and osteogenic properties are optimized.

Provided herein are polymers and polymer synthesis, and particularly the polymers suitable for use in tissue engineering applications.

In one embodiment, provided is a rat periodontitis model to assess the iCPC in vivo. In one embodiment, micro-CT (radiographic bone loss) and immunohistochemistry (IL-1b, IL-6, TNF-a) are utilized to examine the healing and regeneration of periodontal tissue. In one embodiment, statistical analysis is performed using one-way ANOVA and Tukey multiple comparison tests.

In certain embodiment, provided is a composition comprises the iCPC and a dispersant or porogen such as for example, but not limited to water; biological components such as cells, growth factors; bioactive molecules including biopharmaceuticals and drugs or other components such as nano-particulate hydroxyapatite, calcium phosphate or other particles.

In certain embodiment, a porogen is water in an amount of up to 40% of the total weight of the composition. Higher levels of water may be incorporated if an emulsifier is also present in the composition. In such instances water in an amount of up to 80% may be incorporated. Addition of an emulsifier may also help to control pore size and distribution. Any emulsifier could be used but emulsifiers such as block copolymers of polyethylene glycol and polypropylene glycol (Pluronic available from BASF), block copolymers of polysiloxane and polyethylene glycol are preferred for biomedical applications. Commercially available emulsifiers that may be suitable include Symperonic PEF127 and Symperonic PE L101 (Unigema).

In certain embodiments, the disclosed composition may be engineered in either as aqueous or an organic environment and to have an injectable viscosity or to be formed as an in vitro or in vivo solid to suit the application at hand.

In certain embodiments, the disclosed composition further comprises one or more of polycaprolactone diol (400-2000), polycaprolactone triol, poly(lactic acid) diol, polytetraemthylene ether glycol, glycerol with one or more of ethyl 2,6-diisocyanato hexanoate (ethyl lysinediisocyanate), 4,4-methylene bis(phenyl isocyanate), methy 2,6-diisocyanato hexanoate (methyl lysinediisocyanate), hexane diisocyanate, butane diisocyanate. The olefinic functionality is introduced by the use of one or more of isocyanato methacrylate, polyethylene glycol acrylate, glycerol dimethacrylate or isocyanato ethyl methacrylate.

### 4. BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-L The characterization of iCPC@MgO hydrogel conjugates. Synthesis of PXS by acylation of xylitol and succinic acid (A). iCPC polymer continuous polymerization by PXS, caffeic acid, and citric acid (B). The spectrum band changes in the NMR and UV-vis data confirmed the successful introduction of caffeic acid in the iCPC polymer (C-E). iCPC@MgO hydrogels were prepared through a MgO cross-linking reaction (G). The maximum swelling ratio of this hydrogel was reached at about 167% of its weight (H) and over half of the degradation was reached after 21 days (I). Its lap shear adhesive behavior was reinforced compared to fibrin glue (J). The slow-release effect of the Mga'⁰ ions of hydrogel (L).

FIGS. 2A-F Biocompatibility evaluation and osteogenic properties in vitro. No adverse effects on cell viability were observed in the hydrogel group (A), while the cell proliferation was enhanced compared with the control on day 4. Live/dead staining also confirmed that the hydrogel has good biocompatibility, showing the same proliferation trends of CCK8 (B). The superior mineralization effects were also observed by Alizarin Red staining (C); the hydrogel group had more calcium nodules than the control (D). The mRNA and protein level of the Wnt/β-catenin pathway in the hydrogel group was significantly activated, accompanied by a gradual increase in the level of RUNX2 (E/F).

FIGS. 3A-E Anti-inflammatory assessment of hydrogel and computational molecular docking of PXS polymer. ELISA showed a significant decrease in protein levels of IL-6 at 6 h and 12 h, especially in the PXS group (A). The PXS polymer interacted with TLR4, and the oxygen atom of PXS polymer formed 4 hydrogen bonds with Glu81, Glu129, Pyl130, and Val259 (B). qPCR results showed a significant suppression of TLR4 signaling pathway-related genes (C/D).

FIGS. 4A-H Antibacterial efficacy and metabonomics of hydrogel. OD600 values and colony assay showed that the hydrogels inhibited the growth of A.a. and P. gingivalis (P<0.05) (A/B). The intense red fluorescence of dead bacteria increases, and it helps to destroy biofilm formation (D/E). From the metabolomics sequencing results (F/G), the increasing release of FAD is mainly due to the upregulated riboflavin (H).

FIGS. 5A-G Therapeutic effects of periodontal diseases with iCPC@MgO Hydrogel in animal model. Rat periodontitis model establishment (A). Micro-CT results showed that iCPC@MgO hydrogel could reduce radiographic bone loss (P<0.05) and promote periodontal tissue regeneration in rats (B-D). IHC showed more collagen synthesis and osteoclastic activity, and fewer expressed inflammatory factors in the hydrogel-treated group (E). 16S microbiome sequencing found a decrease in the abundance of Firmicutes and Proteobacteria (F/G).

FIG. 6 A schematic diagram showing an embodiment of an application of the disclosed hydrogel.

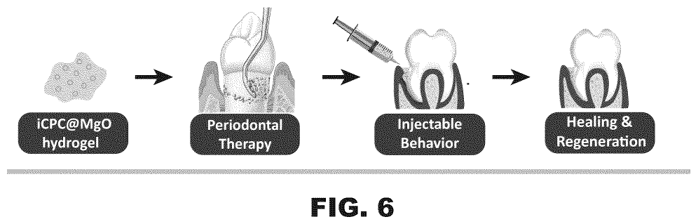

FIG. 7 Spectroscopy characterization of poly (xylitol succinate) (PXS) derivatives.

FIG. 8 Swelling Assay. The right FIG. showed the iCPC@MgO hydrogel was swollen after 24 h. The porous network structure of iCPC@MgO hydrogel was observed after lyophilized.

FIGS. 9A-C Cytotoxicity evaluations of human gingival fibroblast (HGF) cell lines after incubation with various concentrations of all the synthesized materials for 24 h, 48 h, and 72 h (A). Cell viability values of HGF after being treated with different concentrations of lipopolysaccharide (B). The alizarin red staining results under the microscope revealed red calcium nodules after following mineralization induction (C). Data are expressed as mean±S.D. (n=5).

### 4.1 DEFINITION

The following definitions are more general terms used throughout the present application: The term “subject,” as used herein, refers to any animal. In certain embodiments, the subject is a mammal. In certain embodiments, the term “subject”, as used herein, refers to a human (e.g., a man, a woman, or a child).

The terms “administer,” “administering,” or “administration,” as used herein refers to implanting, absorbing, ingesting, injecting, or inhaling, the disclosed polymer or compound.

The terms “treat” or “treating,” as used herein, refers to partially or completely alleviating, inhibiting, ameliorating, and/or relieving the disease or condition from which the subject is suffering.

The terms “effective amount” and “therapeutically effective amount,” as used herein, refer to the amount or concentration of a biologically active agent conjugated to the disclosed polymer, or amount or concentration of the polymer, that, when administered to a subject, is effective to at least partially treat a condition from which the subject is suffering.

“Biocompatible”: The term “biocompatible”, as used herein is intended to describe the polymers that do not elicit a substantial detrimental response in vivo. In certain embodiments, the polymers are “biocompatible” if they are not toxic to cells. In certain embodiments, the polymers are “biocompatible” if their addition to cells in vitro results in less than or equal to 20% cell death, and/or their administration in vivo does not induce inflammation or other such adverse effects. In certain embodiments, biocompatible polymers are also biodegradable.

“Biodegradable”: As used herein, “biodegradable” polymers are those that, when introduced into cells, are broken down by the cellular machinery (e.g., enzymatic degradation) or by hydrolysis into components that the cells can either reuse or dispose of without significant toxic effects on the cells. In certain embodiments, the components do not induce inflammation and/or other adverse effects in vivo. In certain embodiments, the chemical reactions relied upon to break down the biodegradable inventive polymers are enzymatically broken down. For example, the polymers may be broken down in part by the hydrolysis of ester bonds. In certain embodiments, biodegradable polymers are polymers that fully degrade down to their monomeric components under physiological conditions. In certain embodiments, biodegradable polymers are also biocompatible.

The term “pharmaceutically acceptable salt” includes acid addition salts, that is salts derived from a disclosed polymer with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a disclosed polymer contains a substituent with acidic properties, for instance, a carboxylic acid, the term also includes salts derived from bases, for example, sodium, potassium, calcium, magnesium, lithium, and barium salts.

The term “hydrogel,” as used herein, is a polymer which absorbs 10-95 wt % of water (in the presence of an abundance of water).

### 5. DETAILED DESCRIPTION

Conventional injectable hydrogels, composed of various organic and synthetic polymers such as chitosan, polyethylene glycol, and polyvinyl alcohol, have been widely utilized as adhesive hydrogels. Inspired by the adhesive properties of mussels, a dopamine-derived platform has been implemented in these hydrogels. However, these injectable hydrogels still fall short of meeting medical requirements, particularly in terms of accurately matching biological activities such as inflammation resistance and bone regeneration augmentation. Considering the prioritization of biological functions, the selection of raw materials becomes crucial. Among them, xylitol, caffeic acid, and citric acid exhibit unparalleled advantages in terms of their bioactive properties. In this study, we propose the design of a caffeic acid-modified poly-xylitol succinate-based hydrogel (iCPC@Mg) with rapid wet tissue adhesive, degradability, low swelling rate, and injectability, achieved through a metal-ligand connection utilizing magnesium oxide. The incorporation of catechol groups derived from caffeic acid provides the adhesive performance of the hydrogel. Moreover, the iCPC@Mg hydrogel demonstrates therapeutic effects for periodontal diseases, exhibiting outstanding bacteriostatic efficiency against Porphyromonas gingivalis (P.g.) and Aggregatibacter actinomycetemcomitans (A.a.) by stimulating antibiotic synthesis within bacteria and disrupt bacterial cell walls, as well as promoting osteogenesis in human periodontal ligament cells (hPDLSCs) via GSK-30 dependent Wnt/β-catenin pathway. Furthermore, polymer has displayed remarkable anti-inflammatory capabilities by specifically binding to the TLR4 receptor. The combination of these properties, biocompatibility, and environmentally friendly nature, positions the iCPC@Mg hydrogel as a promising candidate for applications in inflammation environments and natural materials.

The regenerative ability of the new polymer can be enhanced by addition of growth factors. Non-limiting examples of growth factors include but are not limited to fibroblast growth factor (FGF), epidermal growth factor (EGF), cilliary neurotrophic factor (CNTF), nerve growth factor (NGF), platelet-derived growth factor (PDGF), vascular endothelial growth factor (VEGF), hepatocyte growth factor (HGF), heparin-binding EGF-like growth factor or HB-EGF and transforming growth factor-alpha. FGF is a preferred growth factor and was used to prove the concept. FGF is a critical growth factor that is representative of the types of GF's associated with the repair and regeneration of tissues. FGF has growth factor receptors (FGFRs), and the main signaling through the stimulation of FGFRs is the RAS/MAP kinase pathway. With their potential biological functions, FGFs have been utilized for the regeneration of damaged tissues, including skin, blood vessel, muscle, adipose, tendon/ligament, cartilage, bone, tooth, and nerve initially been identified as a protein capable of promoting fibroblast proliferation and is now known to comprise 22 members. FGFs exert multiple functions through the binding into and activation of fibroblast.

In one embodiment, provided is an injectable citric acid-PXS-caffeic acid (“iCPC”) polymer.

In certain embodiments, Xylitol can be replaced by other sugar alcohols, such as ribitol, galactitol, and fucitol. Succinyl chloride can be replaced by other dicarbonyl chlorides, such as glutaroyl dichloride and adipoyl dichloride. Citric acid can be replaced by other tricarboxylic acids, such as tricarballylic acid, 2-methyl tricarballylic acid, aconitic acid, and 1,2,4-butane tricarboxylic acid. Caffeic acid can be replaced by dopamine, L-DOPA, D-DOPA, gallic acid, 3,4-dihydroxyhydrocinnamic acid, and tannic acid.

In one embodiment, provided is a polymer formed from one or more monomers of iCPC.

In one embodiment, the iCPC polymer is prepared by a method having the following steps:

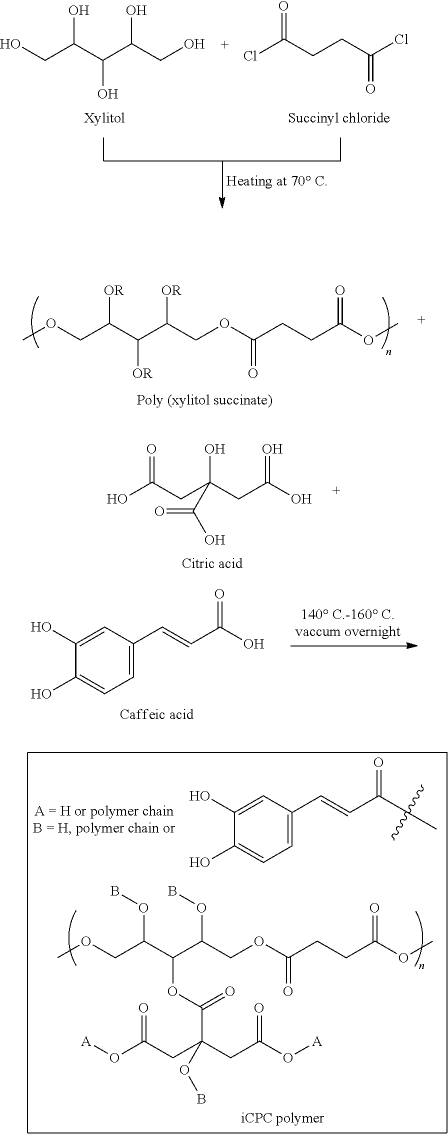

In certain embodiment, n is greater than 1.

In certain embodiments, n is from 2 to 10000 or more. For example, in certain embodiments, the value of n is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 175, 200, 225, 250, 275, 300, 325, 350, 375, 400, 425, 450, 475, 500, 600, 700, 800, 900, 1000, 2000, 3000, 4000, 5000, 6000, 7000, 8000, 9000 or 10000, or is limited to a range defined by any two of said values.

In one embodiment, provided is a composition comprising a polymer formed from one or more monomers of iCPC.

In one embodiment, the method comprises:

Step 1: Synthesis of Poly(xylitol succinate)(PXS), the xylitol and thionyl chloride were heated at 70° C. for 1 hour, then the succinic acid was added into reaction at 70° C. for 2 hrs.

Step 2: Synthesis of iCPC(citric acid-PXS-caffeic acid) polymer. The PXS and citric acid were stirred and heated at 160° C. for 1 hr under nitrogen protection. Then caffeic acid was added overnight reaction.

Step 3: Crosslinking with MgO.

The structure and properties of the polymer can be modified by adjusting the molar ratio of xylitol and succinyl chloride combined in the polymerization reaction. The molar ratio of xylitol to succinyl chloride in the polymer ranges from 0.25 to 1.0. In certain embodiments, they have a molar ratio of 0.5 or 1 (i.e., 1:2 or 1:1 xylitol to succinyl chloride). In one embodiment, the ratio for xylitol, succinic acid, thionyl chloride is 3:3:8.

In certain embodiments, the polymer may further be formed from one or more monomers comprising a catechol-containing species. The catechol containing species can comprise any catechol-containing species not inconsistent with the objects of the present disclosure. In certain embodiments, a catechol-containing species comprises at least one moiety that can form an ester or amide bond with another chemical species used to form a polymer in embodiments. For example, in certain embodiments, a catechol-containing species comprises an alcohol moiety, an amine moiety, a carboxylic acid moiety, or combinations thereof. Further, in certain embodiments, a catechol-containing species comprises a hydroxyl moiety that is not part of the catechol moiety. In certain embodiments, a catechol-containing species comprises dopamine. In certain embodiments, a catechol-containing species comprises L-3,4-dihydroxyphenylalanine (L-DOPA) or D-3,4-dihydroxyphenylalanine (D-DOPA). In still certain embodiments, a catechol-containing species comprises gallic acid or caffeic acid. In certain embodiments, a catechol-containing species comprises 3,4-dihydroxycinnamic acid. Additionally, a catechol-containing species may also comprise a naturally-occurring species or a derivative thereof, such as tannic acid or a tannin. Moreover, in certain embodiments, a catechol-containing species is coupled to the backbone of the polymer or oligomer through an amide bond. In certain embodiments, a catechol-containing species is coupled to the backbone of the polymer through an ester bond.

The physical state of the disclosed polymers is not particularly limited. The polymer can be provided in liquid, solid or semi-solid form. The polymer can be, e.g., injection molded, cast, thermoformed or injection-molded to form objects such as, e.g., sheets, foams, matrices and other three-dimensional objects.

The polymer may take on many different forms, properties, 3-dimensional shapes, and/or sizes. For example, in certain embodiments, the polymer is a bead, microsphere, nanoparticle, pellet, matrix, mesh, gauze, strand, thread, fiber, film, or coating. In certain embodiments, the polymer has a disc-like or spheroidal-like shape. In one embodiment, the polymer is injectable.

In certain embodiments, the polymer is a paste or a wax or has a paste-like or wax-like consistency.

In certain embodiments, the polymer is partially water soluble. In certain embodiments, the polymer is not water soluble. In one specific embodiment, the polymer is partially water soluble.

In certain embodiments, the polymer is a hydrogel.

As used herein, a hydrogel is a polymer which absorbs at least about 10 wt % of water (in the presence of an abundance of water). In certain embodiments, the hydrogel polymer absorbs between about 10 to 100%, 10 to 90 wt %, 10 to 80 wt %, 10 to 70 wt %, 10 to 60 wt %, 10 to 50 wt %, 10 to 40 wt %, 10 to 30 wt %, 10 to 20 wt %, or 10 to 15 wt % of water. In certain embodiments, the hydrogel polymer has an in-vivo half-life of between about 1 week to 6 months. In certain embodiments, the hydrogel has an in-vivo half-life of at least about 1 week, 2 weeks, 3 weeks, 4 weeks, 2 months, 3 months, 4 months, 5 months, or 6 months.

The disclosed polymer can be provided alone or in combination with additional materials in composites. The additional materials include but are not limited to active pharmaceutical ingredients, ceramic structural reinforcement materials, etc.

The unique combination of properties possessed by the disclosed polymer make it suitable for use in a wide variety of applications. The disclosed polymer is particularly well suited for use in tissue regeneration, with strong wet tissue adhesive, osteogenic and antibacterial properties for periodontal regeneration. In certain embodiment, the polymer is suitable for repairing or replacing intervertebral discs, bone, and cartilage.

### 5.1 Polymer Synthesis

The disclosed polymer is provided by polymerization of xylitol and succinyl chloride, citric acid, and caffeic acid. In one embodiment, the polymerization proceeds by a melt condensation synthesis, which is a low cost and simple method without the addition of any toxic catalyst. The polymers can be synthesized with different stoichiometric ratios of xylitol and succinyl chloride, caffeic acid and citric acid with ratios ranging from 1:1:0.1:0.1 to 1:1:1.5:1.5. The properties of the polymers can be modified by modifying the molar ratios of the xylitol and succinyl chloride, caffeic acid, citric acid and/or by modifying the curing time.

In some embodiments, the polymer or oligomer may further be formed from one or more monomers comprising a polycarboxylic acid, such as a dicarboxylic acid, or a functional equivalent of a polycarboxylic acid, such as a cyclic anhydride or an acid chloride of a polycarboxylic acid. In some embodiments, the polycarboxylic acid or functional equivalent thereof can be saturated or unsaturated. In some embodiments, for example, the polycarboxylic acid or functional equivalent thereof comprises maleic acid, maleic anhydride, fumaric acid, or fumaryl chloride.

In some embodiments, the polymer may further be formed from one or more monomers comprising an amino acid, such as an alpha-amino acid. An alpha-amino acid of a polymer described herein, in some embodiments, comprises an L-amino acid, a D-amino acid, or a D,L-amino acid. In some embodiments, an alpha-amino acid comprises alanine, arginine, asparagine, aspartic acid, cysteine, glycine, glutamine, glutamic acid, histidine, isoleucine, leucine, lysine, methionine, proline, phenylalanine, swine, threonine, tyrosine, tryptophan, valine, or a combination thereof. Further, in some embodiments, an alpha-amino acid comprises an alkyl-substituted alpha-amino acid, such as a methyl-substituted amino acid derived from any of the 22 “standard” or proteinogenic amino acids, such as methyl serine.

In some embodiments, the stoichiometric ratio of carboxylic acid groups or derivatives thereof to hydroxyl groups within the monomers used to form the polymer is about 1:1. In some embodiments, the stoichiometric ratio of carboxylic acid groups or derivatives thereof to hydroxyl groups within the monomers used to form the polymer is less than about 1:1. If the stoichiometric ratio is less than about 1:1, the polymer may show defined regions of hydrogen bonding.

Additionally, in some embodiments, a composition comprising a polymer described herein can further comprise a crosslinker. Any crosslinker not inconsistent with the objectives of the present disclosure may be used. In some embodiments, a crosslinker comprises an acrylate or polyacrylate, including a diacrylate. In other embodiments, a crosslinker comprises one or more of 1,3-butanediol diacrylate, 1,6-hexanediol diacrylate, glycerol 1,3-diglyerolate diacrylate, d(ethylene glycol) diacrylate, poly(ethylene glycol) diacrylate, poly(propylene glycol) diacrylate, and propylene glycol glycerolate diacrylate. In still other embodiments, a crosslinker comprises a nucleic acid, including DNA or RNA. In still other instances, a crosslinker comprises a “click chemistry” reagent, such as an azide or an alkyne. In some embodiments, a crosslinker comprises an ionic crosslinker. For instance, in some embodiments, a polymer is crosslinked with a multivalent metal ion, such as a transition metal ion. In some embodiments, a multivalent metal ion used as a crosslinker of the polymer comprises one or more of Fe, Ni, Cu, Zn, Mg, or Al, including in the ⁺² or ⁺³ state.

In addition, a crosslinker described herein can be present in a composition in any amount not inconsistent with the objective of the present disclosure. For example, in some embodiments, a crosslinker is present in a composition in an amount between about 5 weight percent and about 50 weight percent, between about 5 weight percent and about 40 weight percent, between about 5 weight percent and about 30 weight percent, between about 10 weight percent and about 40 weight percent, between about 10 weight percent and about 30 weight percent, or between about 20 weight percent and about 40 weight percent, based on the total weight of the composition.

In some embodiments, the compositions described herein can exhibit a tensile strength of about 1 MPa to about 120 MPa in a dry state as measured according to ASTM Standard D412A, for example of about 2 MPa, 10 MPa, 20 MPa, 30 MPa, 40 MPa, 50 MPa, 60 MPa, 70 MPa, 80 MPa, 90 MPa, or 100 MPa.

In some embodiments, the compositions described herein can exhibit a tensile modulus of about 1 MPa to about 3.5 GPa in a dry state as measured according to ASTM Standard D412A, for example about 1 MPa, about 10 MPa, about 50 MPa, about 100 MPa, about 250 MPa, about 500 MPa, about 750 MPa, about 1 GPa, about 1.5 GPa, about 2 GPa, about 2.5 GPa, about 3 GPa, or about 3.5 GPa.

The compositions described herein can be useful for promoting and/or accelerating periodontal or bone regeneration, including growth, healing, and/or repair as further described herein. It should be understood that one or more compositions described herein can be used in one or more methods of promoting and/or accelerating regeneration described herein, including for growth, healing, and/or repair.

In some embodiments, the compositions described herein useful for promoting periodontal or bone growth can comprise a graft or scaffold. A “graft” or “scaffold”, for reference purposes herein, can refer to any structure usable as a platform or implant for the replacement of missing bone or for promotion of growth of new bone. Moreover, as utilized herein, a “graft” or “scaffold” may be synonymous. For example, a graft or scaffold composition described herein can be used in the repair or replacement of a periodontal or bone defect, or for the promotion of new growth. Further, it is to be understood that grafts or scaffolds consistent with compositions and methods described herein can have any structure or be formed in any shape, configuration, or orientation not inconsistent with the objected of the present disclosure. For example, in some embodiments, a graft or scaffold can be shaped, configured, or oriented in such a manner as to correspond to a defect or growth site to be repaired. In a graft or scaffold, methods described herein can have a shape, configuration, orientation, or dimensions adapted to traverse a gap between the bones to be fused and/or to reinforce a bone growth site. In this manner, particular shapes, sizes, orientations and/or configurations of grafts or scaffolds described herein are not intended to be limited to a particular set or subset of modalities on, within, or adjacent to a bone growth site. A “bone site”, as referenced herein, can be any area in which bone regeneration, bone ossification, bone growth, or bone repair may be desired. In certain non-limiting examples, a bone site can comprise or include a bone defect, a site in which bone has been removed or degraded, and/or a site of desired new bone growth or regeneration, as in the case of a spine or other bone fusion.

Various components of compositions which may form part or all of a graft or scaffold utilized for promoting periodontal or bone regeneration have been described herein. It is to be understood that a composition according to the present disclosure can comprise any combination of components and features not inconsistent with the objectives of the present disclosure. For example, in some cases, a composition forming part or all of a graft or scaffold utilized in a composition described herein can comprise a combination, mixture, or blend of polymers described herein. Additionally, in some embodiments, such a combination, mixture, or blend can be selected to provide a graft or scaffold having any osteo-promoting property, biodegradability, mechanical property, and/or chemical functionality described herein.

Further, one or more polymers described herein can be present in a composition forming part or all of a graft or scaffold utilized in any amount not inconsistent with the objectives of the present disclosure. In some embodiments, a graft or scaffold consists or consists essentially of the one or more polymers described herein. In other instances, a graft or scaffold comprises up to about 95 weight percent, up to about 90 weight percent, up to about 80 weight percent, up to about 70 weight percent, up to about 60 weight percent, up to about 50 weight percent, up to about 40 weight percent, or up to about 30 weight percent polymer, based on the total weight of the graft or scaffold. In some embodiments, the balance of a graft or scaffold described herein can be water, an aqueous solution, and/or an inorganic material as described further below.

In some embodiments, the composition can further comprise an inorganic material. In some embodiments, the inorganic material comprises a particulate inorganic material. Any particulate inorganic material not inconsistent with the objectives of the present disclosure may be used. In some cases, the particulate inorganic material comprises one or more of hydroxyapatite, tricalcium phosphate (including alpha- and beta-tricalcium phosphate), biphasic calcium phosphate, bioglass, ceramic, magnesium powder, pearl powder, magnesium alloy, and decellularized bone tissue particle. Other particular materials may also be used.

In addition, a particular inorganic material described herein can have any particle size and/or particle shape not inconsistent with the objective of the present disclosure. In some embodiments, for instance, a particulate material has an average particle size in at least one dimension of less than about 1000 μm, less than about 800 μm, less than about 500 μm, less than about 300 am, less than about 100 μm, less than about 50 μm, less than about 30 μm, or less than about 10 μm. In some cases, a particular material has an average particle size in at least one dimension of less than about 1 am, less than about 500 nm, less than about 300 nm, less than about 100 nm, less than about 50 nm, or less than about 30 nm. In some instances, a particulate material has an average particle size recited herein in two dimensions or three dimensions. Moreover, a particulate material can be formed of substantially spherical particles, plate-lite particles, needle-like particles, or a combination thereof. Particulate materials having other shapes may also be used.

A particular inorganic material can be present in the compositions (such as a graft or scaffold) described herein in any amount not inconsistent with the objective of the present disclosure. For example, in some cases, a composition utilized as a graft or scaffold described herein comprises up to about 30 weight percent, up to about 40 weight percent, up to about 50 weight percent, up to about 60 weight percent, or up to about 70 weight percent particular materials, based on the total weight of the composition. In some instances, a composition comprises between about 1 and about 70 weight percent, between about 10 and about 70 weight percent, between about 15 and about 60 weight percent, between about 25 and about 65 weight percent, between about 26 and about 50 weight percent, between about 30 and about 70 weight percent, or between about 50 and about 70 weight percent particulate material, based on the total weight of the composition.

Furthermore, these compositions can be made using conventional equipment and techniques known in the art. When preparing dosage forms incorporating the compositions of the disclosure, the ingredients are normally blended with conventional excipients such as binders, lubricants, disintegrants, suspending agents, absorbents, preservatives, surfactants, colorants and suspending agents.

Additional carrier materials can tend to be included, for example water, glycerin, flavoring agents and emulsifiers. Persons of skill in the art will appreciate that other ingredients can be included.

In some embodiments, water is present in the range of between 35% to 85% w/w. In further embodiments, water is present between 50% to 75% w/w.

Glycerin, with or without polyglycitol syrup, can be used to add sweetness, taste, viscosity and texture to embodiments of the present disclosure. The range for glycerin content is between 10% and 30% w/w. In further embodiments, the glycerin content is between 15% and 25% w/w. The range for polyglycitol syrup is between 0% and 15% w/w in some embodiments, and between 5% and 10% w/w in further embodiments.

In some embodiments, the graft or scaffold may be itself a particulate. The particulate graft or scaffold may include or contain a liquid or be substantially “dry” or free of liquid. Moreover, such a liquid that is included in (or mostly excluded from) such as to particular graft or scaffold can be any liquid not inconsistent with the objectives of the present disclosure. In some embodiments, for instance, the liquid is water or an aqueous solution or mixture, such as saline. Moreover, in some embodiments, the liquid can be a carrier liquid for introducing other species to the particulate graft or scaffold. For example, in some embodiments, the liquid comprises one or more biomolecules, bioactive materials, or other biomaterials, as described further below. In some embodiments, the liquid comprises a hyaluronate or hyaluronic acid. In other embodiments, the liquid comprises blood or plasma.

Additionally, the particulate graft or scaffold, in some embodiments, is a paste. More particularly, such a paste can include the particulate graft or scaffold and a liquid (as opposed to being a “dry” material). Such a “paste” can be a viscous or shape-stable material (at standard temperature and pressure conditions) and can have a viscosity suitable for handling or manipulation, such as scooping, with a microspatula. For example, in some embodiments, the paste has a dynamic viscosity of at least 1.0×10⁴ centipoise (cP), at least 5.0×10⁴, or at least 1.0×10⁵. In other embodiments, the paste has a viscosity between about 1.0×10⁴ cP and 1.0×10⁷ cP, between about 1.0×10⁵ cP and 1.0×10⁶ cP, or between 1.0×10⁶ cP and 1.0×10⁷ cP. The liquid component of a paste, in some embodiments, is an isotonic solution, and the paste is a biologically sterile paste. For example, in some embodiments, a paste described herein, can be formed from a salt solution, such as saline, or other biologically active solution such a sodium hyaluronate or blood. In some embodiments, the biologically active solution can comprise additional biological molecules or factors suitable to promote and/or accelerate bone regeneration. For example, the solution can comprise growth factors or signaling molecules, such as osteogenic factors. Non-limiting examples of biological factors that may be used in some embodiments described herein include osteopontin (OPN), osteocalcin (OCN), bone morphogenetic protein-2 (BMP-2), transforming growth factor β3 (TGFβ3), stromal cell-derived factor-1α (SDF-1α), erythropoietin (Epo), vascular endothelial growth factor (VEGF), insulin-like growth factor-1 (IGF-1), platelet derived growth factor (PDGF), fibroblast growth factor (BGF), nerve growth factor (NGF), neurotrophin-3 (NT-3), and glial cell-derived neurotrophic factor (GDNF). Other therapeutic proteins and chemical species may also be used.

In some embodiments, the graft or scaffold described herein is a polymer network. The polymer network can comprise any combination of polymers and/or copolymers described above. Further, in some embodiments, the polymer network comprises an inorganic material (such as a particulate inorganic material). For example, polymers as described above can be cross-linked to encapsulate or otherwise bond to the inorganic material. Cross-linking can be performed, for example, by exposing the polymer to heat and/or UV light. In one embodiment, cross-linking is achieved by ultrasound.

In some embodiments, the compositions described herein deliver the polymer to the site of action (such as a periodontal or bone site) due to their degradation of the composition. In some embodiments, polymer may enhance osteogenic differentiation and tissue regeneration. In some embodiments, polymer may increase osteogenic tissue regeneration by enhancing bioavailability of calcium. In some embodiments, polymer exerts antioxidant and anti-inflammatory action on surrounding cells and/or tissues. In some embodiments, polymer may exert an antimicrobial effect such that it prevents local or implant-associated infection.

In another aspect, the method may further comprise crosslinking the polymer to provide a crosslinked polymer. The polymer may be crosslinked using any of the appropriate methods for crosslinking described herein and as would be readily apparent to those of skill in the art. In some embodiments, the polymer is crosslinked using a crosslinker.

In some embodiments, the method further comprises adding at least one biologically active agent to the formed composition.

### 5.2 Methods of Promoting and/or Accelerating Bone Regeneration

In another aspect, methods of promoting and/or accelerating bone regeneration are described herein. Methods described herein can use one or more compositions described herein. For example, in some embodiments, a method of promoting and/or accelerating periodontal or bone regeneration comprises delivering a composition to a periodontal or bone site. The composition, in some cases, comprises a biodegradable scaffold. Additionally, in some instances, a method described herein further comprises delivering stem cells to the periodontal or bone site.

Methods of promoting and/or accelerating bone regeneration, as described herein, in some embodiments, can further comprise delivering stem cells to the bone site. For example, a graft or scaffold delivered to a bone site consistent with the methods described herein, in some embodiments, can be delivered to a bone site that is seeded with or contains a biofactor or seed cell. In some embodiments, a graft or scaffold can be seeded with a biofactor or cell such as mesenchymal stem cells (MSCs). In certain other embodiments, a graft or scaffold can be delivered to a bone site in addition to or in combination with an autologous bone graft. Biofactors or cells utilized in combination with a graft or scaffold described herein may be isolated or sourced from any host or by any means not inconsistent with the objectives of the present disclosure. For example, in some embodiment, the biofactor or cells can be harvested or isolated from the individual receiving the graft or scaffold. In certain other embodiments, the biofactor or cells can be harvested or isolated from a different individual, such as a compatible donor. In some other cases, the biofactor or cells can be grown or cultured from any individual such as the graft or scaffold recipient or another compatible individual. In certain other cases, the graft or scaffold is unseeded with a biofactor or cell upon disposition within, on, or near the bone site. Non-limiting examples of seed cells that me be used in some embodiments herein include mesenchymal stem cells (MSCs), bone marrow stromal cells (BMSCs), induced pluripotent stem (iPS) cells, endothelial progenitor cells, and hematopoietic stem cells (HSCs). Other cells may also be used. Non-limiting examples of biofactors that may be used in some embodiments described herein include bone morphogenetic protein-2 (BMP-2), transforming growth factor β3 (TGFβ3), stromal cell-derived factor-1α (SDF-1α), erythropoietin (Epo), vascular endothelial growth factor (VEGF), insulin-like growth factor-1 (IGF-1), platelet derived growth factor (PDGF), fibroblast growth factor (BGF), nerve growth factor (NGF), neurotrophin-3 (NT-3), and glial cell-derived neurotrophic factor (GDNF). Other therapeutic proteins and chemical species may also be used.

Methods of promoting and/or accelerating bone regeneration, in some embodiments, can also comprise or include additional steps. Individual steps may be carried out in any order or in any manner not inconsistent with the objectives of the present disclosure. For example, in some embodiments, methods described herein further comprise reestablishing a blood supply to the bone site and/or a biological region adjacent to the bone site. In certain cases, reestablishing a blood supply can comprise or include sealing or suturing biological tissue adjacent to the site. Additionally, in some cases, where blood flow has been artificially restricted at or adjacent to the site, such as by clamping or suction, reestablishing a blood supply can comprise or include releasing or removing the artificial restriction. Further, in some cases, a method of promoting and/or accelerating regeneration can comprise or include increasing one or more of osteoconduction, osteoinduction, osteogenesis, and angiogenesis within the bone site and/or a biological area adjacent to the bone site. Additionally, in some instances, methods further comprise stimulating regeneration of periodontal or bone and/or soft tissue proximate to the periodontal or bone site.

Recruitment of resident mesenchymal stem cells and/or MSCs provided in methods described above can transform or differentiate into osteoblasts at the periodontal or bone site. An intramembranous ossification site can be any developed or developing intramembranous bone tissue in need of bone regeneration.

Moreover, in some embodiments, methods of promoting and/or accelerating regeneration described herein can comprise delivering a graft or scaffold, as described above, before and/or during an early state of osteogenic differentiation at the periodontal or bone site. For example, the scaffold, in some cases, is delivered during early stages of regeneration, such as the proliferation stage and/or matrix maturation stage, occurring after initiation of osteogenic differentiation and prior to maturation.

Moreover, in some embodiments, methods of promoting and/or accelerating regeneration described herein can comprise maintaining the graft or scaffold in the site for a period of time after disposing the graft or scaffold in the growth site. Any period of time not inconsistent with the objective of the present disclosure can be used. For example, in some cases, the graft or scaffold can be maintained for at least 1 month, such as for at least 3 months, at least 6 months, at least 9 months, or at least 12 months. In certain embodiments, a graft or scaffold may degrade or biodegrade within the site. In such embodiments, maintenance of the graft or scaffold can comprise or include maintaining the graft or scaffold until a desired portion of the graft or scaffold has degraded or biodegraded. For example, methods can comprise maintaining the graft or scaffold in the site until at least 50%, at least 60%, at least 70%, at least 80%, at least 90%, at least 95%, or at least 99% of the graft or scaffold has degraded or biodegraded. In certain embodiments, methods can comprise maintaining the graft or scaffold in the site until all or substantially all of the graft or scaffold has degraded or biodegraded. In some embodiments, biodegradation of the graft or scaffold can be measured by measuring the fluorescence intensity at the time of delivery and comparing additional fluorescence intensity measurements at later times to the time of delivery measurement.

Further applications of the compositions described herein include but are not limited to the following: orthopedic tissue engineering materials including composites and porous scaffolds for critical size segmental defect repair and fixation and spinal fusion and films for periosteum repair and barrier functionality; porous scaffolds for wound dressing applications; antibacterial materials; antioxidant materials; antiresorptive materials for osteoporosis treatment; self-healing materials; and injectable materials for void filling and fracture fixation.

In certain embodiments, the composition further comprises an antioxidant, pharmaceutically active agent, biomolecule, or cell.

In certain embodiments, the composition is stable for 4-6 months.

In one embodiment, a method of promoting and/or accelerating periodontal or bone regeneration is provided comprising delivering a composition described herein to a periodontal or bone site. In certain embodiments, the composition is delivered before and/or during a proliferation stage of osteogenesis at the periodontal or bone site. In certain embodiments, the method further comprises delivering stem cells to the periodontal or bone site.

In certain embodiments, the bone site is an intramembranous ossification site. In certain embodiments, the bone site is an endochondral ossification site.

In one embodiment, provided is a kit for promoting and/or accelerating periodontal regeneration comprising a composition described herein.

### 5.3 Methods of Use

The polymers may be useful in drug delivery (e.g., delivery of antibiotics, drugs, bioactive agents); as an injectable drug delivery system for mechanically taxing environments (such as periodontal sites or within joints) where the material may release a drug in a controlled manner without being compromised by a dynamic or static external environment; in ligament repair; as osteo-synthesis material (e.g., screws); in intervertebral disc repair; in soft tissue repair; in bone tissue repair; use of biodegradable particles, tubes, spheres, strands, threads, coiled strands, films, sheets, fibers, meshes, and the like; in surgical glue and adhesives; for injectables; for vascular grafts; microfabrication applications (capillary networks, diagnostics) for tissue engineering (i.e., bladder, bone, brain, nerve, skin, cardiac tissue, ligament, cartilage, tendon, genital, muscle, artery, veins, kidney, pancreas, liver, intestine, stomach, and other tissues); and as an injectable (e.g., to aid in cosmetic and/or surgical procedures).

The present disclosure provides a method for the prevention or treatment of a disease, condition, or disorder in a subject comprising administering the polymer or polymer composition as described above and herein, to a subject in need thereof.

In certain embodiments, a therapeutically effective amount of the polymer or composition is administered. As used herein, a “therapeutically effective amount” of an inventive polymer or composition is an amount that can achieve a desired therapeutic and/or prophylactic effect. A “therapeutically effective amount” is at least a minimal amount of the polymer or composition which is sufficient for preventing, ameliorating, reducing, delaying, or diminishing severity of a disease, disorder, or condition in a subject.

Exemplary diseases, conditions, or disorders which may be treatable by the polymers or pharmaceutical compositions of the present disclosure includes: periodontal disease, diseased tissue, bone or muscle injuries, bone breakage or fracture, joint conditions, arthritis, sepsis, necrosis, bone disorders, dental conditions, and the like.

In one embodiment, the disorder to be treated is periodontal disease, also known as gum disease. In certain embodiment, the disorder to be treated is gingivitis and/or periodontitis In one embodiment, the disorder is a set of inflammatory conditions affecting the tissues surrounding the teeth. In its early stage, called gingivitis, the gums become swollen and red and may bleed. It is considered the main cause of tooth loss for adults worldwide. In its more serious form, called periodontitis, the gums can pull away from the tooth, bone can be lost, and the teeth may loosen or fall out. Halitosis (bad breath) may also occur.

Periodontal disease is generally due to bacteria in the mouth infecting the tissue around the teeth. Factors that increase the risk of disease include smoking, diabetes, HIV/AIDS, family history, high levels of homocysteine in the blood and certain medications. Diagnosis is by inspecting the gum tissue around the teeth both visually and with a probe and X-rays looking for bone loss around the teeth.

In certain embodiments, the polymer, or pharmaceutical composition comprising the polymer, is administered to the subject by any known means, e.g., injection, periodontal, transdermal, orally, parenterally, subcutaneously, intracutaneously, intradural, subdural, epidural, by surgically implantation, absorption, the polymer or pharmaceutical composition.

In certain embodiments, the polymer is a component of a biomedical device or implant. In certain embodiments, the polymer is a polymer film or coating on an implant. In certain embodiments, the polymer is an implant. In certain embodiments, the polymer implant is a polymer matrix.

In certain embodiments, the polymer is injected or implanted into a subject. In certain embodiments, the pre-polymer to the polymer (i.e., prior to polymerization) is injected or implanted into a subject. In certain embodiments, the pre-polymer is injected or surgically implanted, and polymerized in vivo.

In one embodiment, the polymer is surgically implanted or injected into a subject on or near diseased or damaged tissue. In certain embodiments, the polymer implant aids in the in-growth of surrounding healthy tissue to the diseased area.

In certain embodiments, the polymer further includes a biologically active agent. Biologically active agents include any substance used as a medicine for treatment, prevention, delay, reduction or amelioration of a disease, condition, or disorder, and refers to a substance that is useful for therapy, including prophylactic and therapeutic treatment. Abiologically active agent also includes a compound that increases the effect or effectiveness of another compound, for example, by enhancing potency or reducing adverse effects of the other compound.

In certain embodiments, the biologically active agent is cleaved from the polymer upon enzymatic hydrolysis. In certain embodiments, the biologically active agent, upon release or cleavage, participates in treating a condition, disease, or disorder in a subject.

The following examples are intended to illustrate the scope of the disclosure and to enable reproduction and comparison. They are not intended to limit the scope of the disclosure in any way.

### 6. EXAMPLES

### 6.1 Materials and Methods

Xylitol, succinate, citric acid, and caffeic acid were purchased from Dieckmann (HK) Chemical Co., Ltd., and magnesium oxide (342793-250G), alizarin red staining (A5533), Alkaline Phosphatase Assay Kit (MAK447-1KT) were the products of Sigma-Aldrich. LIVE/DEAD Cell Imaging Kit (Cat. No. R37601), LIVE/DEAD™ BacLight™ (L7012) were purchased from Thermofisher Scientific. CCK8 kit (C0038) was purchased from Beyotime Biotechnology and the Human IL-6 Elisa kit was provided by Raybiotech.

Synthesis of Poly (xylitol succinate) (PXS): The PXS conjugate was synthesized as previously described^{[18]}. First, the xylitol (3 mmol) and thionyl chloride (8 mmol) were heated at 70° C. for 1 h, then the succinic acid was added to the reaction at 70° C. for 2 h. The crude mixture was neutralized with ammonium bicarbonate (1 mol L⁻¹) after being dissolved in DI water and cooled down to room temperature, the product was next extracted with dichloromethane. The solution was passed through a plug of silica gel and afterward, evaporation of the solvent to get a transparent viscous product of PXS. The molecular weight of PXS was measured using gel permeation chromatography (GPC).

Synthesis of iCPC (citric acid-PXS-caffeic acid) Polymer: The PXS (1 mmol) and citric acid (2 mmol) were placed in a double-necked flask, and the reaction mixture was stirred and heated at 160° C. for 1 h under nitrogen protection. Then caffeic acid (0.3 mmol) was added to the mixture and the temperature was reduced to 140° C. overnight. The produced prepolymer was purified by dialysis using the dialysis membrane (MWCO 500-1000 Da) in DI water for 7 days and subsequence freeze-dried before use. The raw materials and polymers were dissolved in DI water to perform UV-vis absorption spectroscopy at a wavelength of 200-500 nm to get the conjugation efficiency of iCPC polymer.

Preparation ofiCPC@Mg composite Hydrogels: The iCPC polymer was first dissolved in the mix solution (ethanol/DI water=4:1) to form a 40 wt % of polymer solution, then 20 wt % MgO was added in DI water and the dispersion was quickly shaken by hand. The preparation of iCPC@Mg hydrogel was through the mixing of the iCPC polymer mix solution and MgO dispersion with a volume ratio of 2:1. The pre-hydrogel solution was sonicated 4 hours to allow for crosslinking with an average temperature of −45° C. After freeze-drying the hydrogel, the Fourier-transform infrared (FTIR) spectroscopy was carried out to confirm the presence of diverse functional groups of raw materials, iCPC polymer, hydrogel, and the structure of the molecule. The 1H NMR (400 MHz) spectra were recorded in CDCl3 for PXS, DMSO-d6 for caffeic acid and D₂O for citric acid, PXS+citric acid, iCPC, iCPC@Mg hydrogel.

Soft hard tissue Adhesive Strength of iCPC Polymer and iCPC@Mg Hydrogel Under Saliva Conditions: The adhesion strengths of iCPC polymers and iCPC@Mg hydrogels were investigated by tensile-stress and lap shear experiments using a universal testing machine under a 500N load cell with stretch rate of 1 cm min⁻¹ at room temperature. Fresh porcine skin and bone were cut into a 4 mm×4 mm section, then cemented on the surface of ‘T’ shaped sheet. The tissues were wetted by soaking in artificial saliva before use. 500 μL of the iCPC polymer and iCPC@Mg hydrogel were added and spread over the surface of tissues, then the two strips were brought in contact and allowed to cure for 5 minutes. Each sample was tested five times in parallel.

Rheological measurements: The iCPC@Mg Composite Hydrogel and its pre-solution were measured by using a dynamic rheometer (TA Instrument, U.S.A.), equipped with a parallel plate (diameter=40 mm, gap=50 μm). At least triplicates were carried out for each rheological measurement.

Swelling Ratio, Degradation Profiles and Water Content of iCPC@Mg Composite Hydrogel: 4 mg of dried hydrogel was immersed in distilled water for 8 h. After predetermined periods, the supernatant was removed from the swollen samples, and the resulting hydrogels were weighed (W_{d}) at 2, 4, 8, 12, 24 and 48 h. To evaluate the degradation of the iCPC@Mg composite hydrogel, the dried hydrogel specimens were accurately weighed (W₀) and soaked in PBS solution at 37° C. in shaking for 2, 5, 10, 15, and 20 days. The remaining hydrogel was freeze-dried after discharging the PBS at the prescribed time to obtain the residual mass (Wₜ). Mass loss and the swelling ratio were calculated using equation as respectively [³²]:

$$\begin{matrix}Mass loss ( \% ) = \frac{W 0 - Wt}{W 0} \times 100 \% \\ Swelling ratio ( \% ) = \frac{Wd}{W 0} \times 100 \% \\ Water content ( \% ) = ( {mass}_{swollen} - {mass}_{dried} ) / {mass}_{swollen} \times 100 \%\end{matrix}$$

In Vitro Cytotoxicity Study: The cell proliferation of the hydrogels was studied by quantitative CCK-8 cytotoxicity assay. Human periodontal ligament cells (hPDLSCs) were cultured in α-minimum essential medium supplemented with 10% FBS and 1% penicillin/streptomycin, 5% CO₂, and 37° C. After the iCPC@Mg hydrogel was smeared evenly into 96-well microculture plates, a density of 3×10⁴ cells/100 μL was seeded in each well. The cells were further incubated for 1, 2, and 4 days. At present, the mediums were replaced by 100 μL 10% CCK-8 reagent for 1 h. Finally, the OD value of each group at 450 nm was measured by a microplate reader. hPDLSCs were seeded in confocal dishes with 1×10⁴ cells per dish, the Calcein AM/iodide Live-Dead cell double staining was also carried out on direct visualization of the cell viability using 1:100 dilution stock solution.

Osteoinductive Capacity Assay: The osteogenic capacity of iCPC@Mg composite hydrogel was determined by alkaline phosphatase staining (Alp) and alizarin red staining. Osteogenic differentiation of hPDLSCs was first induced by 0.1 μmol/L dexamethasone, 50 g/ml VC, and 10 mmol/L sodium β-glycerophosphate containing medium and cultured in six-well plates. 0.2% Alizarin red was applied to stain the formation of mineralized nodules after 21 days, and the alkaline phosphatase activity was performed using the Alkaline Phosphatase Assay Kit on day 4 and day 7.

Antibacterial Properties: The groups of iCPC@Mg composite hydrogel were set the same as before against the Porphyromonas gingivalis ATCC 33277 and Aggregatibacter actinomycetemcomitans ATCC 29522. The two bacteria were cultured in BHI medium at 37° C. anaerobically to reach a final density of 1×10⁵ CFU mL¹ and prepared the hydrogel culture medium solutions as well the PBS mixed bacteria suspensions in a 96-well plate reaching the difference concentrations (4096, 2048, 1024, 512, 256 g/ml). After incubation for 1 to 8 hours, the bacterial growth was monitored using OD₆₀₀. Then, bacteria suspensions were properly diluted and used for coating the plates, followed by anaerobic cultured at 37° C. for 4 h, the number of colony-forming units at 5× per replicate were taken photos at the Zeiss Axio Observer Al and counted after cultured at 24 h.

To test the destruction of bacterial biofilm, the iCPC@Mg hydrogel was first injected into the bottom of cell climbing slices. P.g. and A.a were anaerobically in cultured in TSB medium at the surface of hydrogel and allowed to form biofilms 7 days after. The viability of bacteria attached to iCPC@Mg hydrogel surfaces was assessed by live/dead staining under Zeiss LSM 900 confocal laser scan microscope after 24 h.

A transmission electron microscope (TEM) was conducted to observe the microstructure changes of bacteria, after co-cultured hydrogel with bacteria, the specimens were certificated at 10,000 rpm for 10 min to allow bacterial aggregates to settle and then fixed with 2.5% glutaraldehyde at 4° C. overnight. The aggregates were washed by PBS three times and dehydrated using gradient alcohol (50, 70, 90, 100%), then embedded in resin, sectioned, stained, and observed under a Transmission electron microscope (JEM 1400 PLUS, USA).

In Vivo Periodontitis Study: All animal procedures were approved by the institutional animal ethics guidelines of The University of Lanzhou. The Ligature-induced periodontitis model was adapted from a previous study [⁶° ]. Each male Sprague Dawley rat at 8 weeks old was properly anesthetized and randomly allocated into different experimental groups, then a 2-0 silk ligature was tied around the maxillary first molar of rats to induce periodontitis for four weeks. After removing the ligature, iCPC@Mg hydrogel was injected into the atrophy gingival groove once every three days for 0.2 ml on each edge of the periodontal bone defect and cured within one month. The periodontitis rats without any treatments were used as blank control, traditional commercial minocycline hydrochloride gel (Perio) served as a positive control group. Maxillary segments and surrounding periodontal tissues were fixed via 4% paraformaldehyde after sacrificed animals with carbon dioxide. For histology analysis, specimens were decalcified in 10% EDTA for one month, then embedded in paraffin wax and performed staining the inflammation markers (IL-1, IL-6, TNF-α), Masson's trichrome staining, TRAP staining. Bone mineral density, volume analysis, and bone imaging were scanned by a MicroCT machine.

### 6.2 Example

### 6.2.1 Polymerization of Xylitol and Succinyl Chloride (PXS)

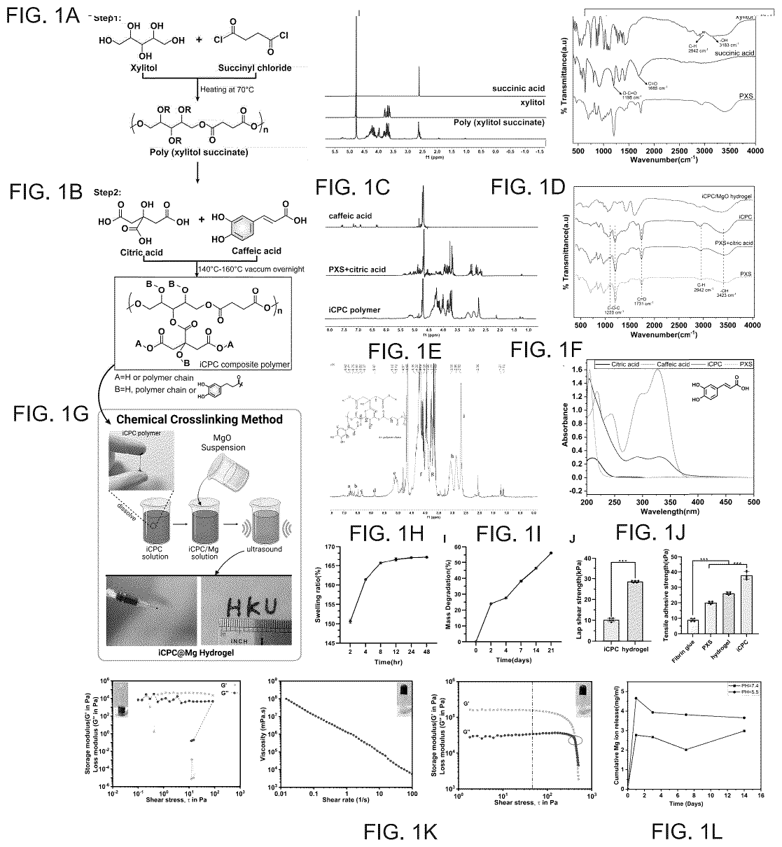

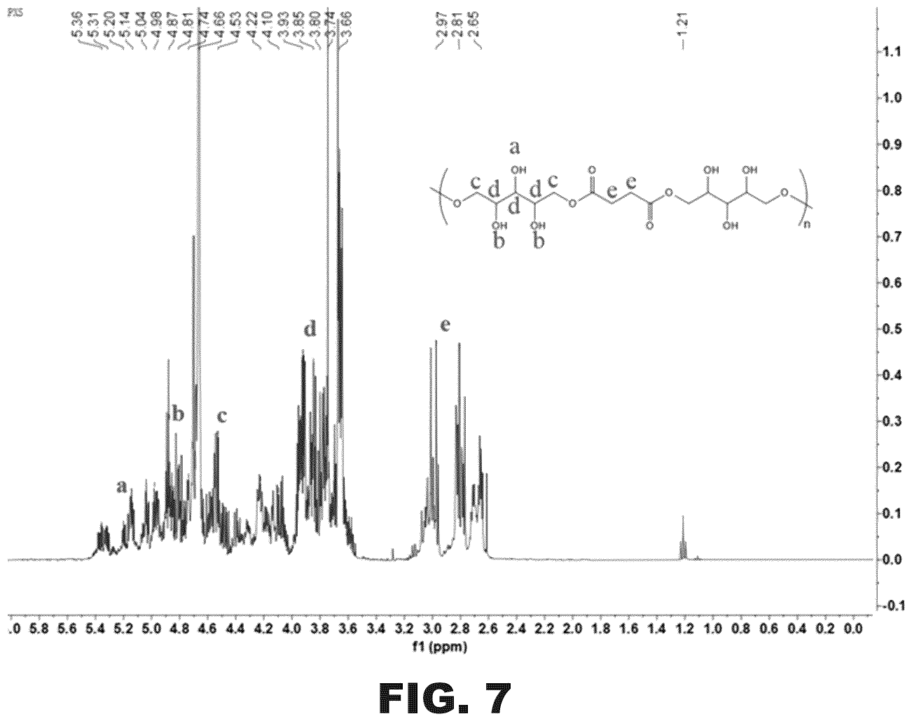

The synthetic route of iCPC polymer involves two steps, as illustrated in FIGS. 1A and B which the poly xylitol succinate (PXS) has the same polymerization method as previous literature^{[18]}. The 1H nuclear magnetic resonance (NMR) and FTIR spectrum of PXS and two starting materials are provided in FIG. 1A. The 1H NMR spectrum of PXS is divided into three regions, zone from 4.8 to 5.4 ppm corresponds to the methine protons from xylitol, zone from 4.0 to 5.0 ppm can be assigned to hydrogel from xylitol protons, zone from 2.5 to 3.1 ppm corresponds to the methylene protons of succinic acid (FIG. 7). The peaks of succinic acid found at 1685 cm-1 and 1198 cm⁻¹ can be attributed to C═O and O—C═O band stretching, and xylitol shows its characteristic OH stretching at 3183 cm⁻¹. After polymerization, peaks were observed at 1220 cm⁻¹, 2942 cm⁻¹′ and 3423 cm-1 that respectively exhibited the characteristic bands of C—O—C, C—H, and O—H. The molecular weight of PXS measured by gel permeation chromatography (GPC) as the Mn values are 687 kDa, and Mw is 697 kDa. These results agree with the previous reports that a short chain of polyester is synthesized from xylitol and succinic acid.

6.2.2 Synthesis and Characterization of iCPC Polymers

Caffeic acid-poly (xylitol succinate)-citric acid (iCPC) was prepared via an esterification reaction of PXS followed by dehydration condensation and chain extension to form a network structure. The schematic diagram and proton nuclear magnetic resonance spectrum of this compound are shown in FIG. 1B. From NMR results (FIG. 1C and FIG. 1E), the proton signals of caffeic acid involved in polymerization show peaks at 5.8 to 6.8 ppm: the peaks at 6.82 and 5.87 ppm correspond to the proton signals of the olefin, multiple signals in the range of 6.37-6.70 ppm correspond to the benzene ring protons of caffeic acid. The multiple signals around 5.0-5.3 ppm correspond to the proton signals of the free hydroxyl groups and some tertiary methyl groups of the xylitol in the polymer. The strong signals at 3.5-4.4 ppm correspond to the proton of the tertiary methyl-CH-groups connected to various oxygen atoms with xylitol in the polymer, which confirms that xylitol is the main constituent unit. The two main signals at 2.5-3.2 ppm correspond to the two methylene-CH₂-protons of the succinic acid molecules in the polymer, and the single peak at 2.69 ppm corresponds to the methylene-CH₂-proton of the citric acid involved in the polymerization (FIG. 1E).

From FTIR results, the O—H stretching band intensity increased present at 3423 cm-1 for iCPC polymer in the spectrum indicating the increasing catechol group from caffeic acid (FIG. 1D). In addition, we speculated that the addition of magnesium ions during the crosslinking might cause the horizontally shifted spectrum. The absorbance changes of iCPC at each wavelength of the UV-vis spectra are shown in FIG. 1F, the two characteristics peaks from caffeic acid on the iCPC polymer are observed around 280 and 330 nm, it also exhibited a marked absorption peak at 210 nm from the poly xylitol succinate. The spectrum and absorption band changes in the NMR and FTIR data confirm the successful introduction of caffeic acid in iCPC composite polymer.

6.2.3 Gelation of iCPC@Mg Conjugates and Characterizations

In the way of polymer materials crosslinking, divalent transition metal ions have been proved to form and act as crosslinks with 4 arms of polyethylene glycol-dopamine in viscoelastic gel networks, such as Zn²⁺, Ni²⁺, Cu^{2+ [38]}. The cross-linking reaction mechanism and procedures are depicted in FIG. 1G, the upturned trial suggesting that iCPC@Mg hydrogel was successfully fabricated through a MgO involved metal-ligand crosslinks reaction after an ultrasound of the mixture solution. At the same time, the injectable behavior of hydrogel was visualized by a 1 ml needle. The reason injectable hydrogel is regarded as a great candidate for dental materials is the uneven shape of the tooth and easy operation in the clinic^{[39]}.

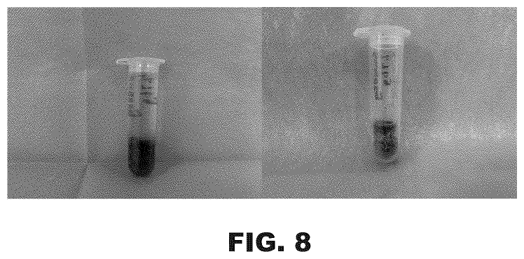

The porous network structure was obtained for the lyophilized sample of hydrogel (FIG. 8). Consideration of narrow spaces across the periodontal tissues, the hydrogel has a high demand for swelling resistance. As confirmed with a quantitative survey, the maximum swelling ratio of this hydrogel was reached at about 167% of its own weight and the swollen gel plateaued from 12 hours onward (FIG. 1H). We also found that the hydrogel was capable of rapidly degrading 24% within 2 days, and over half of the degradation rate was reached after 21 days (FIG. 1I). A slow degradation of hydrogel most likely contributes to its biological effects. Although the tensile adhesive strength of the hydrogel was not higher of iCPC polymer, its lap shear adhesion behavior was reinforced compared to that of iCPC polymer (FIG. 1J). The high adhesiveness of hydrogel is likely attributed to the iCPC composite polymer that it derived from the catechol groups provided by caffeic acid, and the interaction between MgO with catechol groups or carboxyl groups of iCPC polymer to form the surface bonding or hydrogen bonding^{[32]}. The adhesive characteristic of hydrogel helps optimize the antibacterial effect of medical applications^{[40]}. Compared with the periodontitis-applied hydrogel from previous literature, the iCPC@Mg hydrogel in our study showed an enhancement of adhesion strength, which was about 6 times of poly (ethylene imine) based hydrogel^{[41]}.

6.2.4 iCPC@Mg Composite Hydrogel Enables Enhancement of hPDLSCs Osteogenic Differentiation by Activating Wnt/β-Catenin Pathway

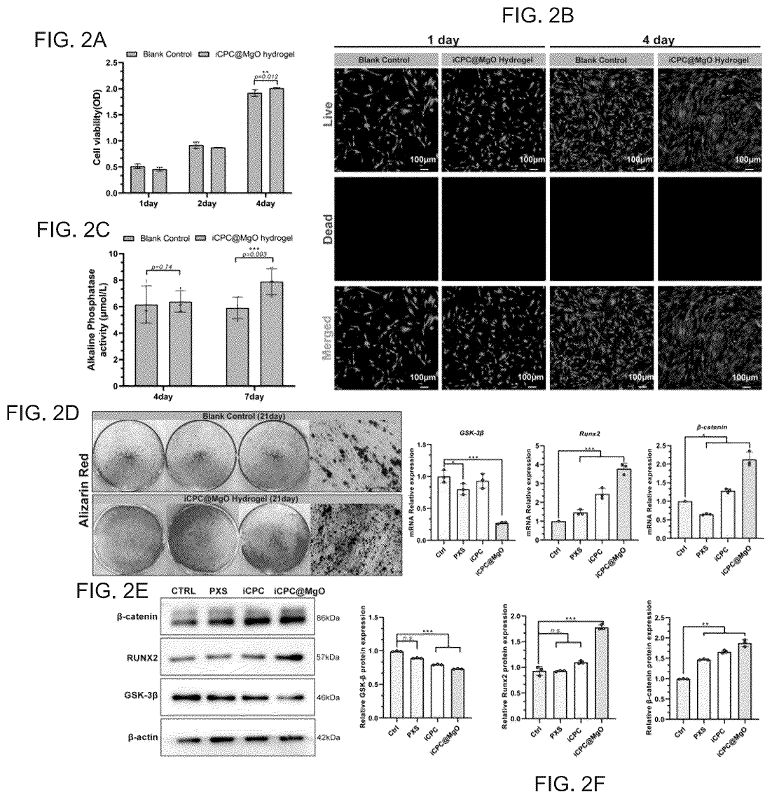

Beyond the adhesive characteristics of this injectable hydrogel, another important aspect of the dental application is cytocompatibility which has a therapeutic role for periodontitis by promoting the repair of periodontitis-related alveolar bone defects. Before further assessing the osteogenic differentiation ability of hydrogel, the sterilized hydrogel was directly injected into the dishes to measure the biocompatibility of human periodontal ligament cells (hPDLSCs) made with CCK8 assay and live/dead staining. After 1 and 2 days of incubation, no adverse effects on cell viability were observed in the hydrogel group (FIG. 2A), while the cell proliferation promotion happened compared with the control on day 4. Live/dead staining also confirmed the hydrogel has good biocompatibility by showing the same proliferation trends of CCK8 (FIG. 2B). That might arise from the natural biosafety characteristics of our starting materials: caffeic acid is naturally produced through the metabolism of vegetables or plants [⁴²], and as a five-carbon sugar alcohol, xylitol is also mainly used for the food industry.

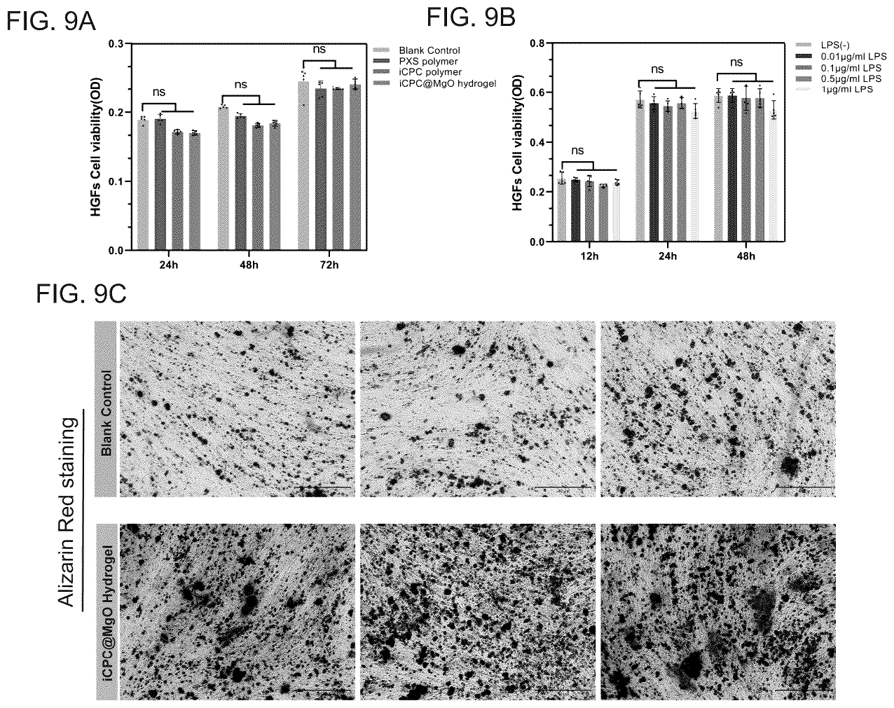

Bone regeneration is in urgent demand nowadays and remains challenging for alveolar bone loss related to periodontitis^{[43]}. To further evaluate the osteogenesis activities in vivo, the hPDLSCs cells were first osteogenic induction under specific culture conditions, alkaline phosphatase evaluation at 7 days of incubation displayed a significantly increased release in the hydrogel group indicating that our hydrogel enhanced the early stage of osteogenic capacity (FIG. 2C). The superior mineralization effects were also supported by Alizarin Red staining (FIG. 2D), the iCPC@Mg hydrogel cocultured hPDLSCs cells had more calcium nodules than the control group (FIG. 9). Accordingly, the expression of β-catenin and RUNX2 was significantly increased in MgO addition, together with a significantly lower expression of GSK-30 ion channels when compared to the other three groups (FIG. 2E and FIG. 2F). These results were further validated at the protein level. As reported in previous studies, the pro-osteogenic performance can be achieved by the release of Mg²⁺ during the degradation process of hydrogels^{[44]}. Therefore, with magnesium in hand, the MgO applied for the crosslinking process of hydrogel might enhance its osteogenic differentiation properties to periodontal ligament cells.

### 6.2.5 Poly Xylitol Succinate Inhibits the IL-6 Release by Binding the Domain of Human Tol-Like Receptor 4 Under Lipopolysaccharide-Induced High Inflammatory State

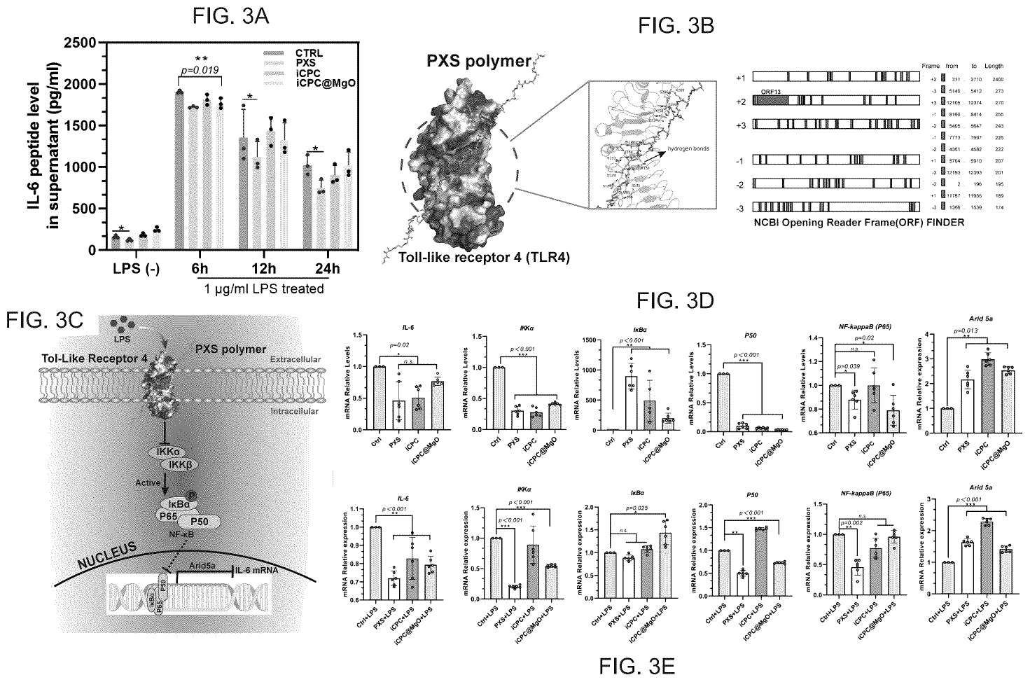

As the gingival inflammatory reaction of periodontitis generally started from the fibroblast cells, the human gingival fibroblast cells (HGFs) were chosen for the anti-inflammation evaluation of hydrogel (FIG. 9A). For co-treatment experiments, a significant reduction in IL-6 was confirmed to activate the state of HGFs inflammation after being treated with LPS (FIG. 3A). However, there was down-regulation of IL-6 at 6 h in the iCPC@Mg hydrogel group compared with the model, especially the PXS polymer group significantly inhibited the protein release of IL-6 at 6, 12, and 24 h, even without the pro-inflammatory effect brought by LPS, it's also in line with the down-regulation of IL-6 mRNA which indicated that the PXS polymer might be the primary functional component in controlling inflammation.

To further explore the mechanism of PXS polymer resisting inflammation, we performed a molecular docking simulation analysis of the interaction between polymer and human Toll-like receptor 4 (TLR4) protein. Theoretically, the Toll-like receptor 4 could be firstly activated by LPS binding to exhibit inflammation effects and promote IL-6 release by stimulating the NF-κB pathway^{[45]}. The computational docking methodology is a rapid and reliable method in predicting protein-additive interactions and then helps in finding out the bio-based polymer-additive complexes with corresponding superior properties^{[46]}. As illustrated in molecular docking results, the oxygen atom of the PXS polymer formed four hydration hydrogen bonds with Glu81, Glul29, Pyl130, and Val259 when docked with the crystal structure of the TLR4 which is shown in yellow lines (FIG. 3B), which suggests that TLR4 has a good binding force with PXS polymer because of the hydrogen bonds are powerful intermolecular forces. The docking free energy of the TLR4 molecule reached −50.4288 kilocalories per mole (kcal/mol). Thus, we speculate that the anti-inflammatory function of PXS polymer may contribute to its binding to TLR4 receptors with high affinity in a competitive manner. To demonstrate that binding would suppress the activation of the nuclear factor-κB (NF-κB) pathway, we studied the effect of PXS polymer on the gene expression of NF-κB inhibitor α (IκBα), p65 (RelA), p50 (NFκB1), IκB Kinase α (IKKα) and Arid 5a. As shown in FIG. 3C-E, PXS polymer inhibited the expression of IKKα and led to a decrease in P50 expression which in turn suppresses the NF-κB pathway. Typically, the activation of NF-κB not only relies on an inducible degradation of IκBα^{[47]} but also mediates by a powerful negative feedback loop of IκBα that may remove NFκB from the nucleus^{[48,49]}. We have performed the IκBα protein phosphorylation levels quantification in order to get a deeper insight into the reaction mechanism.

The figure showed that the PXS polymer significantly suppresses NFκB pathway by activating the phosphorylation of IκBα which may result in IL-6 suppression.

6.2.6 iCPC@Mg Composited Hydrogel Effectively Eliminates the A. actinomycetemcomitans and P. gingivalis by Stimulating Antibiotic Synthesis within Bacteria and Disrupting Bacterial Membrane

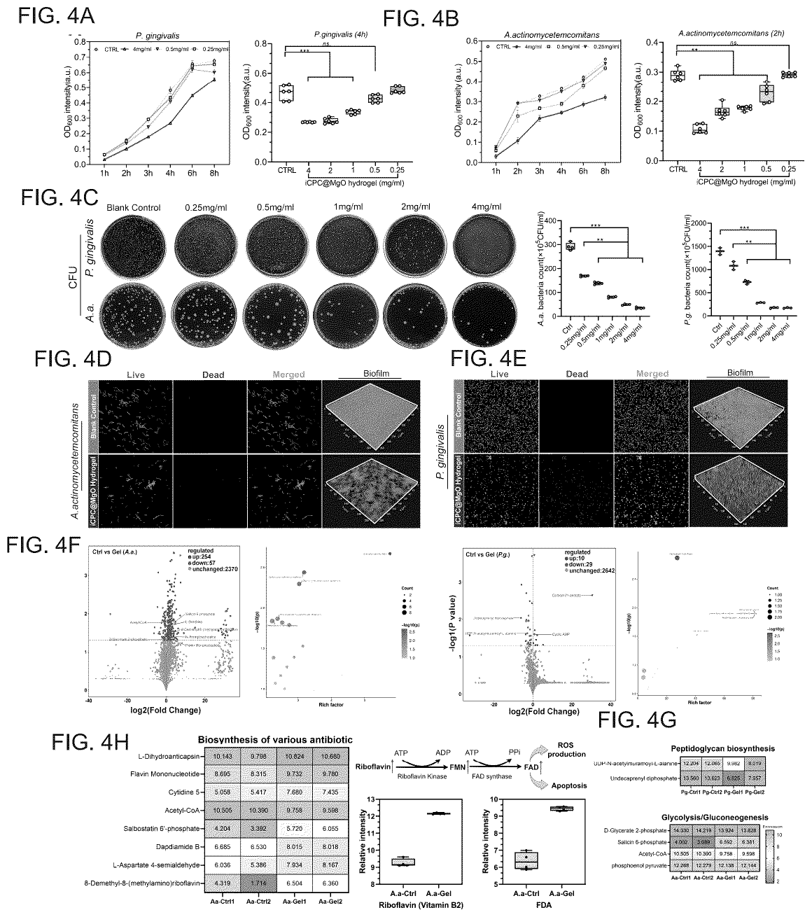

We selected the two dominant bacteria from subgingival flora that cause periodontal tissue infections, Porphyromonas gingivalis and Aggregatibacter actinomycetemcomitans to evaluate the bacteriostatic efficiency of hydrogel^{[50]}. The minimal inhibitory concentration (MIC) of iCPC@Mg hydrogel against A. actinomycetemcomitans and P gingivalis is 500 g mL⁻¹ and 1000 g mL⁻¹ (FIG. 4A and FIG. 4B). Approximately half of the antibacterial concentrations of the hydrogel is 4 mg mL⁻¹ against A.a. and P.g. after being cultured for 2 or 4 hours. In addition, Live/Dead staining was performed to distinguish dead from live bacteria to evaluate the bactericidal properties of the iCPC@Mg hydrogel. As shown in FIG. 4C, the intense red fluorescence of dead bacteria increases indicating that a substantial number of bacteria were killed after co-cultured bacteria with hydrogel. Due to the development of periodontitis modulated by biofilm, thus destroying bacterial biofilm is the first step towards effective control of inflammatory reaction^{[51]}. The green fluorescence intensity of the P.g. and A.a. biofilms treated with hydrogel is notably lower than the blank control group (FIG. 4C-E), similar trends were observed in the biofilm quantitatively results, the bio-thickness and biovolume treated with hydrogel was significantly lower than the group without treatment which proved iCPC@Mg hydrogel exhibited the good plaque biofilm destroy performance by preventing the formation of biofilm.

Several metabolites exhibit differences between the two groups from the present metabolomics sequencing results explaining the new mechanisms of antibacterial action of iCPC@Mg hydrogels. Specifically, in hydrogel groups (FIG. 4F), there is a significant increase in metabolites related to antibiotic production within A.a bacteria, namely N-Formylmethionine (fMet), L-Ornithine, and Flavin Mononucleotide (FMN). Conversely, metabolites associated with decreased glycolysis, such as D-Glycerate 2-phosphate, are observed. The increasing antibiotic synthesis could potentially enhance antimicrobial activity^{[52]}. For example, FMN is a precursor for the synthesis of a class of antibiotics called aminoglycosides, which allows the antibiotics to bind to the ribosomes in bacteria and interfere with protein synthesis, ultimately leading to bacterial cell death^{[51]}. From the specific metabolites concentration analyzed results (FIG. 4), the increasing release of FAD is mainly due to the upregulated riboflavin. When glycolysis is decreased in bacteria, several consequences can occur such as reduced energy production, impaired bacteria growth and proliferation, or altered metabolic flux^{[54,55]}. Our results also showed that different bacteria tend to exhibit distinct reactions to the same hydrogels. In the case of iCPC@Mg, the notable influence on peptidoglycan synthesis and biofilm synthesis in P.g. bacteria was observed, indicating its antibacterial effects through the disruption of the bacterial membrane. This disruption of the biological membrane was visualized using TEM electron microscopy, as shown in FIG. 4C. Decreased peptidoglycan synthesis can lead to a weakened cell wall, rendering the bacteria more susceptible to mechanical stress and environmental pressures^{[56]}.

Two of the antimicrobial potential raw materials were applied into iCPC@Mg hydrogel that is xylitol reduced the dental plaque accumulation by interfering with the energy cycle of bacteria^{[57]}, and caffeic acid well defined antibacterial activity may be associated with damage of membrane integrity due to the polyphenolic structures can interact with the bacterial membrane^{[58]}, among the other examined polyphenols such as gallic acid and vanillic acid, caffeic acid possessed better antibacterial activity^{[59]}. These bacteriostatic properties of iCPC@Mg hydrogel indicate that poly (xylitol succinate) not only can form hydrogel through caffeic acid modification, but also exert overall biological functional benefits as an ensemble.

6.2.7 Healing Effects of Periodontal Diseases with iCPC@Mg Composited Hydrogel In Vivo

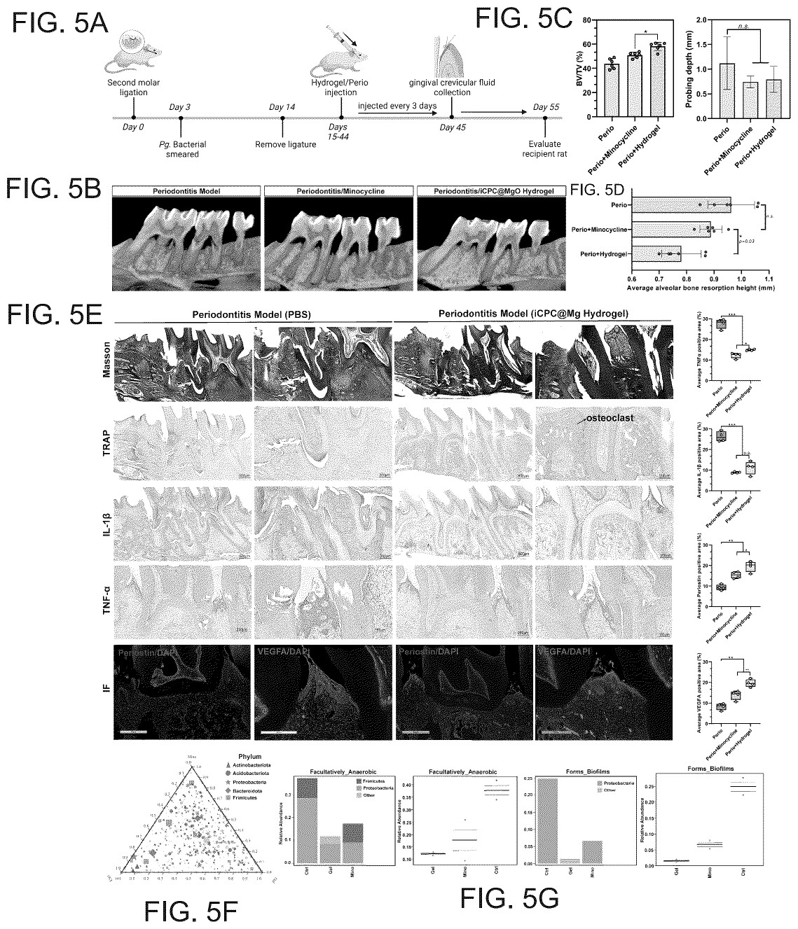

To assess the efficacy of hydrogel in vivo, we first established and characterized the model of topical periodontitis via bilaterally ligated on the maxillary second molar to 4 weeks (FIG. 5A). Periodontitis parameters including probing depth and gingival bleeding were measured in response to periodontal soft and hard tissue destruction. Our micro-CT results showed a lower distance between the alveolar bone crest (ABC) to the cementoenamel junction (CEJ) in rats treated with iCPC@Mg hydrogel group compared with the antibiotic-exposure positive control group for one month (FIG. 5B), BV/TV increased at 57.5% in hydrogel group. Additionally, the TRAP staining appears a significant increase in the number of osteoclasts of the alveolar bone, and the fluorescence intensity of the Runx2-labeled protein was 3.2 times higher compared with other groups. Besides the Soft tissue injury in terms of inflammation was evaluated using hematoxylin-eosin, Masson's trichrome, and immunohistochemical (IHC) staining. Immunohistochemical results indicated the hydrogel reduces the accumulation of pro-inflammatory factors located in gingival tissues, such as IL-6 and IL-10 (FIG. 5D and FIG. 5E). The highest degree of collagen deposition surrounding alveolar bone was observed in the hydrogel group via Masson staining.

Furthermore, using the microbiome sequencing technology (16S) of the V3-V4 region, we next evaluated if the iCPC@Mg hydrogel affects the spatial structuring of the subgingival microbiota. The results of the ternary abundance analysis indicated that our hydrogel has no negative impact on the richness of subgingival bacterial communities (FIG. 5G), which can preserve the balance of subgingival microbiota. Additionally, it shows a relative decrease in the abundance of Bacteroidetes, Firmicutes, and Proteobacteria after comparing control and minocycline hydrochloride groups.

### 6.3 Conclusions

In summary, we have successfully developed a whole specific safety injectable hydrogel system with multifunctional properties for the treatment of periodontitis. Unlikely the commonly used antibiotics or previously reported synthetic polymer-based hydrogels, our approach prioritizes the safety and self-provided biological value of the raw materials. By focusing on the development of a more biocompatible and safer iCPC polymer, which has eliminated the need for additional bioactive peptides or ingredients. Our hydrogel system not only exhibits excellent anti-inflammatory, antibacterial, and osteogenic properties in vitro as well as in vivo but also provides a solid foundation for future other applications.

Exemplary products, systems and methods are set out in the following items:
1. A polymeric compound represented by the structure of formula (I):

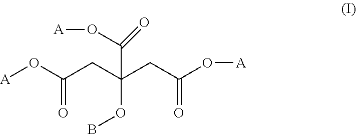

wherein
   each A is independently H or a poly-(xylitol succinate) optionally substituted by B; and
   each B is independently H, a poly-(xylitol succinate), or a moiety comprising a catechol group;
wherein
   at least one of A and B is a poly-(xylitol succinate); and
   at least one of A and B is a moiety comprising a catechol group;
or a pharmaceutically-acceptable salt thereof.
2. The compound of item 1, wherein the poly-(xylitol succinate) is represented by the structure of formula (1a), (1b) or (1c):

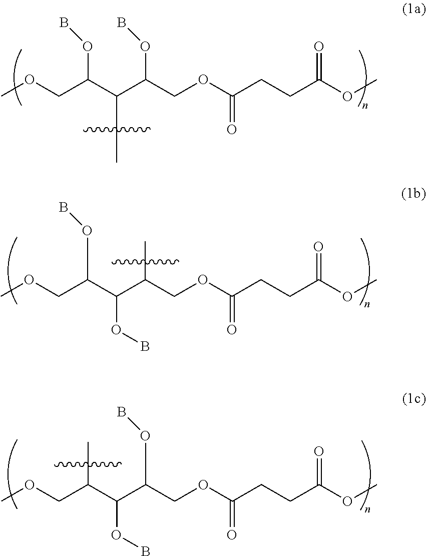

wherein
   n is an integer from 1 to 500; and
   [symbol] is a point of attachment to formula (I).
3. The compound of anyone of the preceding items, wherein B is a moiety comprising a catechol group.
4. The compound of anyone of the preceding items, wherein B is derived from caffeic acid, and is represented by formula (2)

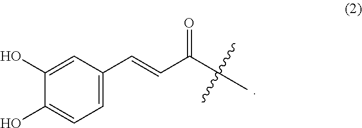 [SMILES: *C(=O)/C=C/c1ccc(O)c(O)c1]

5. A polymeric compound represented by the structure of formula (II):

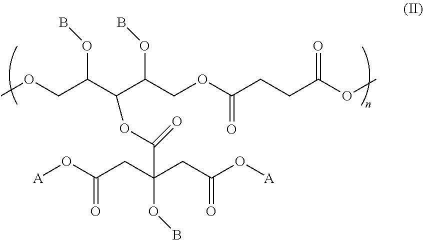

wherein
   each A is independently H or a poly-(xylitol succinate) optionally substituted by B;
   each B is independently H, a poly-(xylitol succinate), or a moiety comprising a catechol group; and
   n is an integer of 1-500;
wherein
   at least one of A and B is a poly-(xylitol succinate); and
   at least one of A and B comprises a catechol group;
or a pharmaceutically-acceptable-salt thereof.
6. The compound of anyone of the preceding items, wherein the poly-(xylitol succinate) is represented by the structure of formula (1a), (1b) or (1c):

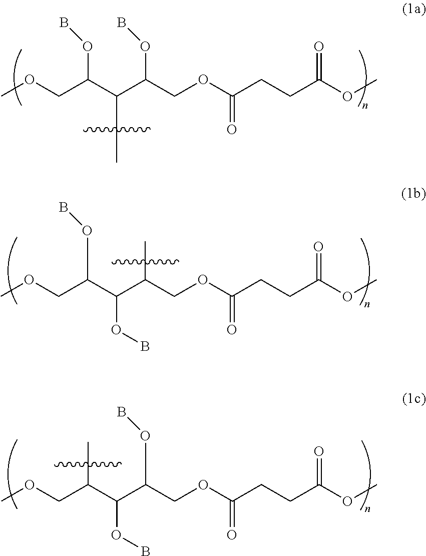

wherein
   n is an integer from 1 to 500; and
   [symbol] is a point of attachment to formula (I).
7. The compound of anyone of the preceding items, wherein B is a moiety comprising a catechol group.
8. The compound of anyone of the preceding items, wherein B is derived from caffeic acid, and is represented by formula (2)

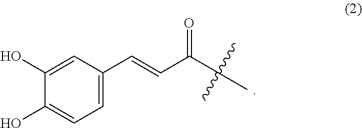 [SMILES: *C(=O)/C=C/c1ccc(O)c(O)c1]

9. A hydrogel comprising the compound of anyone of the preceding items, wherein the compound is cross-linked with metal particles.
10. The hydrogel of item 9, wherein the metal is an alkaline earth metal.
11. The hydrogel of anyone of the preceding items, wherein the metal is Mg or Ca.
12. A process for preparing the compound of anyone of the preceding items, comprising the step of reacting poly-(xylitol succinate) with citric acid and a moiety comprising a catechol group.
13. The process of anyone of the preceding items, wherein the moiety comprising a catechol group is caffeic acid.
14. The process of anyone of the preceding items, wherein the poly-(xylitol succinate) is prepared by reacting xylitol with succinic acid or an activated derivative thereof.
15. The process of anyone of the preceding items, wherein the activated derivative is succinyl chloride.
16. The process of anyone of the preceding items, further comprising heating the reaction.
17. The process of anyone of the preceding items, further comprising reacting the polymeric compound with a metal oxide.
18. The process of anyone of the preceding items, wherein the metal is an alkaline earth metal.
19. The process of anyone of the preceding items, wherein the metal is Mg or Ca.
20. The process of anyone of the preceding items, wherein the metal oxide is MgO or CaO.
21. A process for preparing a hydrogel, comprising reacting the polymeric compound of anyone of the preceding items with a metal oxide.
22. The process of item 21, wherein the metal oxide is MgO or CaO.
23. The compound of anyone of the preceding items that is injectable.
24. An injectable composite polymer comprising caffeic acid, polyxylitol succinate (PXS) and citric acid prepared by the method having the steps of:

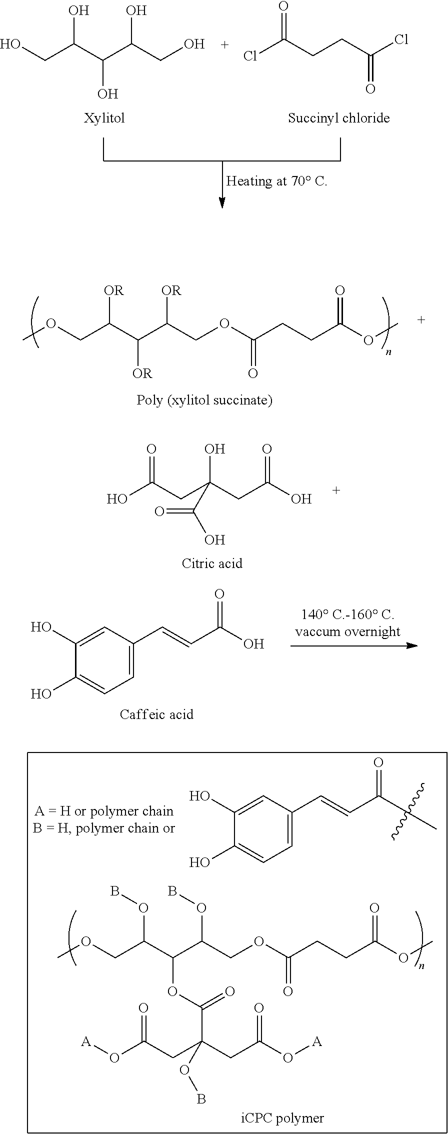

wherein n is 1-500.
25. A method of making an injectable caffeic acid/polyxylitol succinate (PXS)/citric acid composite polymer (iCPC) hydrogel comprising the steps of:
   (i) Esterifying xylitol with succinyl chloride to form polyxylitol succinate (PXS) polymer;
   (ii) Combining PXS and caffeic acid (CFA) in the presence of citric acid in a one-pot synthesis forming a pre-polymer solution; and
   (iii) Crosslinking the pre-polymer solution with MgO.
26. The compound of anyone of the preceding items wherein the polymer is a hydrogel.
27. The compound of anyone of the preceding items characterized by a proton nuclear magnetic resonance (¹H NMR) spectra of FIG. 1E.
28. The compound of anyone of the preceding items having anti-bacterial effects and osteogenic properties.
29. The compound of anyone of the preceding items which is biodegradable or biostable.
30. A composition comprising the polymer compound of anyone of the preceding items and a pharmaceutically acceptable carrier.
31. The composition of item 31 further comprising one or more of the group consisting of radical inhibitor, sensitizer, promoter, dispersant, porogen, catalyst, biological components, bioactive molecules, hydroxyapatite, calcium phosphate and surfactant.
32. The composition of anyone of the preceding items wherein the composition is a dental or periodontal cement composition.
33. The composition of anyone of the preceding items wherein the composition is an aqueous emulsion for the delivery of cells, growth factors or bioactive molecules including biopharmaceuticals and drug.
34. An implant comprising the polymer compound of anyone of the preceding items.
35. A method of preventing or treating a disorder comprising administering the polymer compound of anyone of the preceding items to a subject in need thereof.
36. The method of item 36 wherein the disorder is a periodontal disease.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

[1] C. Xian, Q. Yuan, Z. Bao, G. Liu, J. Wu, Chinese Chemical Letters 2020, 31, 19.
[2] Y. Chen, C. Sun, J. Cao, Y. Wu, B. A.-O. Cui, J. Ma, H. Wang, Polymers (Basel) 2022.
[3] Y. A.-O. Go, C. A.-O. X. Leal, Chem Rev 2021, 121(22).
[4] S. G. Alamdari, A. Alibakhshi, M. de la Guardia, B. Baradaran, R. Mohammadzadeh, M. Amini, P. Kesharwani, A. Mokhtarzadeh, F. Oroojalian, A. Sahebkar, Adv Healthc Mater 2022, 11, e2200526.
[5] D. Xie, J. Guo, M. Mehdizadeh, R. T. Tran, R. Chen, D. Sun, G. Qian, D. Jin, X. Bai, J. Yang, J Mater Chem B 2015.
[6] D. A. Hickman, C. L. Pawlowski, U. D. S. Sekhon, J. Marks, A. S. Gupta, Adv Mater 2018.
[7] P. Grenier, V. Chénard, N. Bertrand, J Control Release 2023.
[8] F. Yang, X. A.-O. Niu, X. Gu, C. Xu, W. Wang, Y. Fan, ACS Appl Mater Interfaces 2019.
[9] Z. Li, G. Li, J. Xu, C. Li, S. Han, C. Zhang, P. Wu, Y. Lin, C. Wang, J. A.-O. Zhang, X. Li, Adv Mater 2022.
[10] L. Xiao, M. Feng, C. Chen, Q. Xiao, Y. Cui, Y. A.-O. Zhang, Adv Mater 2023.
[11] A. A.-O. Lin, C. A.-O. Giuliano, A. Palladino, K. A.-O. John, C. Abramowicz, M. L. Yuan, E. L. Sausville, D. A. Lukow, L. Liu, A. A.-O. X. Chait, Z. A.-O. Galluzzo, C. A.-O. Tucker, J. A.-O. Sheltzer, Sci Transl Med 2019.
[12] N. Li, L. Xie, Y. Wu, Y. Wu, Y. Liu, Y. Gao, J. Yang, X. Zhang, L. Jiang, Mater Today Bio 2022.
[13] Z. Tu, Y. Zhong, H. Hu, D. A.-O. X. Shao, R. Haag, M. Schirner, J. Lee, B. Sullenger, K. A.-O. Leong, Nat Rev Mater 2022.
[14] J. A.-O. Luyendyk, J. G. Schoenecker, M. J. Flick, Blood 2019.
[15] K. Zheng, Q. Gu, D. Zhou, M. Zhou, L. Zhang, Smart Materials in Medicine 2022, 3, 41.
[16] J. P. Bruggeman, C. J. Bettinger, C. L. E. Nijst, D. S. Kohane, R. Langer, Adv Mater 2008, 20, 1922.
[17] W. Dong, T. Li, S. Xiang, P. Ma, M. Chen, Polymers 2013, 5, 1339.
[18] J. P. Bruggeman, C. J. Bettinger, C. L.E. Nijst, D. S. Kohane, R. Langer, Adv Mater 2008, 20, 1927.
[19] Y. F. Wu, E. Salamanca, I. W. Chen, J. N. Su, Y. C. Chen, S. Y. Wang, Y. S. Sun, N. C. Teng, W. J. Chang, Front Nutr 2022, 9, 882636.
[20] E. Park, H. S. Na, S. M. Kim, S. Wallet, S. Cha, J. Chung, J Periodontol 2014, 85, e212.
[21] D. Clark, E. Kotronia, S. E. Ramsay, Periodontol 2000 2021, 87, 143.
[22] Z. Sheikh, N. Hamdan, Y. Ikeda, M. Grynpas, B. Ganss, M. Glogauer, Biomater Res 2017, 21, 9.
[23] M. F. Tolba, H. A. Omar, S. S. Azab, A. E. Khalifa, A. B. Abdel-Naim, S. Z. Abdel-Rahman, Crit Rev Food Sci Nutr 2016, 56, 2183.
[24] T. Xu, H. Zhu, R. Liu, X. Wu, G. Chang, Y. Yang, Z. Yang, Front Immunol 2022, 13, 1005430.
[25] A. Stahli, C. U. Maheen, F. J. Strauss, S. Eick, A. Sculean, R. Gruber, Int J Oral Sci 2019, 11, 6.
[26] Y. K. Fu, B. J. Wang, J. C. Tseng, S. H. Huang, C. Y. Lin, Y. Y. Kuo, T. C. Hour, C. P. Chuu, J Biomed Sci 2022, 29, 16.
[27] H. Mude, P. A. Maroju, A. Balapure, R. Ganesan, J. Ray Dutta, Food Chem 2022, 374, 131830.
[28] S. H. Zainal, N. H. Mohd, N. Suhaili, F. H. Anuar, A. M. Lazim, R. Othaman, J Mater Res Technol 2021, 10, 935.
[29] D. Gyawali, P. Nair, Y. Zhang, R. T. Tran, C. Zhang, M. Samchukov, M. Makarov, H. K. W. Kim, J. Yang, Biomaterials 2010, 31, 9092.
[30] A. Brown, S. Zaky, H. Ray, Jr., C. Sfeir, Acta Biomater 2015, 11, 543.
[31] Y. Chen, W. Sheng, J. Lin, C. Fang, J. Deng, P. Zhang, M. Zhou, P. Liu, J. Weng, F. Yu, D. Wang, B. Kang, H. Zeng, ACS Appl Mater Interfaces 2022, 14, 7592.
[32] Y. Xiong, Z. Lin, P. Bu, T. Yu, Y. Endo, W. Zhou, Y. Sun, F. Cao, G. Dai, Y. Hu, L. Lu, L. Chen, P. Cheng, K. Zha, M. A. Shahbazi, Q. Feng, B. Mi, G. Liu, Adv Mater 2023, 35, e2212300.
[33] X. Lu, S. Shi, H. Li, E. Gerhard, Z. Lu, X. Tan, W. Li, K. M. Rahn, D. Xie, G. Xu, F. Zou, X. Bai, J. Guo, J. Yang, Biomaterials 2020, 232, 119719.
[30] S. Lilienthal, A. Fischer, W. C. Liao, R. Cazelles, I. Willner, ACS Appl Mater Interfaces 2020, 12, 31124.
[33] D. Laurencin, N. Almora-Barrios, N. H. de Leeuw, C. Gervais, C. Bonhomme, F. Mauri, W. Chrzanowski, J. C. Knowles, R. J. Newport, A. Wong, Z. Gan, M. E. Smith, Biomaterials 2011, 32, 1826.
[36] W. Qiao, K. H. M. Wong, J. Shen, W. Wang, J. Wu, J. Li, Z. Lin, Z. Chen, J. P. Matinlinna, Y. Zheng, S. Wu, X. Liu, K. P. Lai, Z. Chen, Y. W. Lam, K. M. C. Cheung, K. W. K. Yeung, Nat Commun 2021, 12, 2885.
[37] Y. Wang, Z. Geng, Y. Huang, Z. Jia, Z. Cui, Z. Li, S. Wu, Y. Liang, S. Zhu, X. Yang, W. W. Lu, J Mater Chem B 2018, 6, 6615.
[38] S. C. Grindy, R. Learsch, D. Mozhdehi, J. Cheng, D. G. Barrett, Z. Guan, P. B. Messersmith, N. Holten-Andersen, Nat Mater 2015, 14, 1210.
[39] U. Shirbhate, P. Bajaj, Cureus 2022, 14, e29248.
[40] Y. Guo, M. Wang, Q. Liu, G. Liu, S. Wang, J. Li, Theranostics 2023, 13(1).
[41] X. Zhao, Y. Yang, J. Yu, R. Ding, D. Pei, Y. Zhang, G. He, Y. Cheng, A. Li, Biomaterials 2022, 282, 121387.
[42] V. Lionetti, B. S. Tuana, V. Casieri, M. Parikh, G. N. Pierce, Eur Heart J 2019, 40(7).
[43] Y. Chen, H. Wang, Q. Yang, W. Zhao, Y. Chen, Q. Ni, W. Li, J. Shi, W. Zhang, L. Li, Y. Xu, H. Zhang, D. Miao, L. Xing, W. Sun, Theranostics 2022, 12, 1074.
[44] B. Wen, Y. Dai, X. Han, F. Huo, L. Xie, M. Yu, Y. Wang, N. An, Z. Li, W. Guo, NP J Regen Med 2023, 8, 11.
[45] K. K. Nyati, K. Masuda, M. M. Zaman, P. K. Dubey, D. Millrine, J. P. Chalise, M. Higa, S. Li, D. M. Standley, K. Saito, H. Hanieh, T. Kishimoto, Nucleic Acids Res 2017, 45, 2687.
[46] F. Abookleesh, F. E. S. Mosa, K. Barakat, A. Ullah, Polymers (Basel) 2022, 14.
[47] T. Henkel, I. Machleidt T Fau—Alkalay, M. Alkalay I Fau—Krönke, Y. Krönke M Fau—Ben-Neriah, P. A. Ben-Neriah Y Fau—Baeuerle, P. A. Baeuerle, 1993.
[48] H. E. Dembinski, K. Wismer, J. D. Vargas, G. W. Suryawanshi, N. Kern, G. Kroon, H. J. Dyson, A. Hoffmann, E. A. Komives, Proc Natl Acad Sci USA 2017, 114, 1916.
[49] A. Hoffmann, M. L. Levchenko A Fau—Scott, D. Scott Ml Fau—Baltimore, D. Baltimore, Science 2022, 298.
[50] G. Hajishengallis, T. Chavakis, Nat Rev Immunol 2021, 21, 426.
[51] N. Li, L. Xie, Y. Wu, Y. Wu, Y. Liu, Y. Gao, J. Yang, X. Zhang, L. Jiang, Mater Today Bio 2022, 16, 100360.
[52] M. M. Zhang, Y. Wang, E. L. Ang, H. Zhao, Nat Prod Rep 2016, 33, 963.
[53] E. M. Darby, E. Trampari, P. Siasat, M. S. Gaya, I. Alav, M. A. Webber, J. M. A. Blair, Nature Reviews Microbiology 2023, 21, 280.
[54] M. A. Herman, B. B. Kahn, J Clin Invest 2006, 116, 1767.
[55] A. J. F. Egan, J. Errington, W. Vollmer, Nature Reviews Microbiology 2020, 18, 446.
[56] B. A.-O. Walnerhanssen, A. A.-O. Meyer-Gerspach, C. Beglinger, M. A.-O. Islam, Crit Rev Food Sci Nutr 2020, 60(12).
[57] M. A.-O. Kepa, M. Miklasinska-Majdanik, R. A.-O. Wojtyczka, D. Idzik, K. Korzeniowski, J. Smolen-Dzirba, T. A.-O. Wa̧sik, Biomed Res Int 2018, 15.
[58] M. Friedman, J Agric Food Chem 2014.
[59] J. Tian, C. Liu, X. Zheng, X. Jia, X. Peng, R. Yang, X. Zhou, X. A.-O. Xu, J Dent Res 2020, 99(7).
[60] C. Zhang, T. Li, C. Zhou, L. Huang, Y. Li, H. Wang, P. Duan, S. Zou, L. Mei, Int J Oral Sci 2020, 12, 38.

What is claimed:

1. A polymeric compound represented by the structure of formula (I): 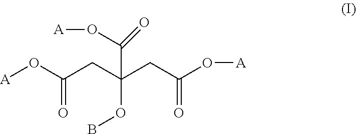
wherein
each A is independently H or a poly-(xylitol succinate) optionally substituted by B; and
each B is independently H, a poly-(xylitol succinate), or a moiety comprising a catechol group;
wherein
at least one of A and B is a poly-(xylitol succinate); and
at least one of A and B is a moiety comprising a catechol group;
or a pharmaceutically-acceptable salt thereof.

2. The compound of claim 1, wherein the poly-(xylitol succinate) is represented by the structure of formula (1a), (1b) or (1c): 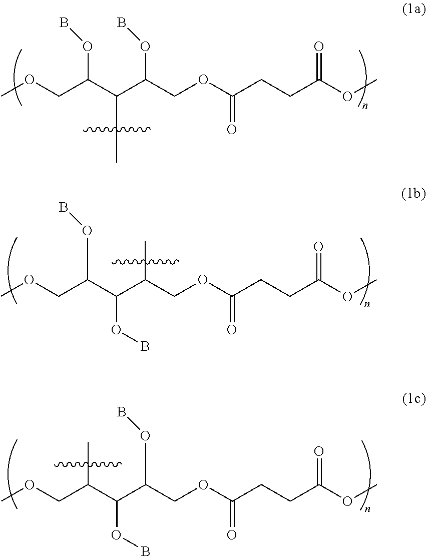
wherein
n is an integer from 1 to 500; and [symbol]
is a point of attachment to formula (I).

3. The compound of claim 1, wherein B is a moiety comprising a catechol group.

4. The compound of claim 3, wherein B is derived from caffeic acid, and is represented by formula (2)

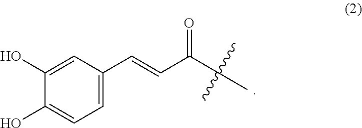 [SMILES: *C(=O)/C=C/c1ccc(O)c(O)c1]

5. A polymeric compound represented by the structure of formula (II): 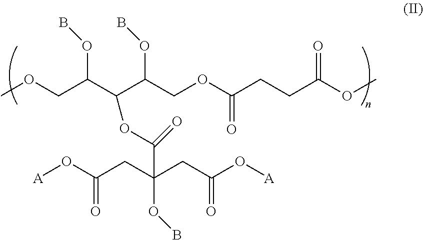
wherein
each A is independently H or a poly-(xylitol succinate) optionally substituted by B;
each B is independently H, a poly-(xylitol succinate), or a moiety comprising a catechol group; and
n is an integer of 1-500;
wherein
at least one of A and B is a poly-(xylitol succinate); and
at least one of A and B comprises a catechol group;
or a pharmaceutically-acceptable-salt thereof.

6. The compound of claim 5, wherein the poly-(xylitol succinate) is represented by the structure of formula (1a), (1b) or (1c): 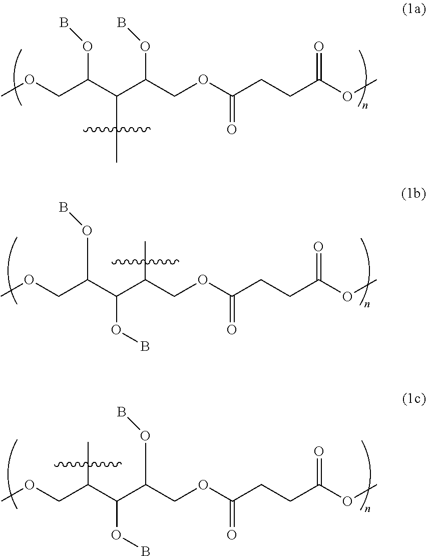
wherein
n is an integer from 1 to 500; and [symbol]
is a point of attachment to formula (I).

7. The compound of claim 5, wherein B is a moiety comprising a catechol group.

8. The compound of claim 7, wherein B is derived from caffeic acid, and is represented by formula (2)

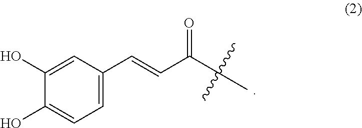 [SMILES: *C(=O)/C=C/c1ccc(O)c(O)c1]

9. A hydrogel comprising the compound of claim 1, wherein the compound is cross-linked with metal particles.

10. The hydrogel of claim 9, wherein the metal is an alkaline earth metal.

11. The hydrogel or claim 10, wherein the metal is Mg or Ca.

12. A process for preparing the compound of claim 1, comprising the step of reacting poly-(xylitol succinate) with citric acid and a moiety comprising a catechol group.

13. The process of claim 12, wherein the moiety comprising a catechol group is caffeic acid.

14. The process of claim 12, wherein the poly-(xylitol succinate) is prepared by reacting xylitol with succinic acid or an activated derivative thereof.

15. The process of claim 14, wherein the activated derivative is succinyl chloride.

16. The process of claim 12, further comprising heating the reaction.

17. A process for preparing a hydrogel, comprising reacting the compound of claim 1 with a metal oxide.

18. The process of claim 17, wherein the metal oxide is MgO or CaO.

19. A composition comprising the compound of claim 1 and a pharmaceutically acceptable carrier, wherein the pharmaceutically acceptable carrier is an aqueous emulsion for the delivery of cells, growth factors or bioactive molecules and wherein the bioactive molecules comprise one or more of the group consisting of radical inhibitor, sensitizer, promoter, dispersant, porogen, catalyst, biological components, bioactive molecules, hydroxyapatite, calcium phosphate and surfactant.

20. The composition of claim 19 wherein the composition is a dental or periodontal cement composition.

21. An implant comprising the polymer compound of claim 1.

22. A method of preventing or treating a disorder comprising administering the compound of claim 1 to a subject in need thereof.

23. The method of claim 22 wherein the disorder is a periodontal disease.
